SEQUENCE LISTING (58 sequences)

SEQ ID NO: 1 - protein, 179 aa, synthetic construct
  source 1..179: note = T33-51 protein cage, Subunit A
MFTRRGDQGETDLANRARVGKDSPVVEVQGTIDELNSFIGYALVLSRWDDIRNDLFRIQNDLFVLGEDVSTGGKGRTVTMDMIIYLIKRSVEMKAEIGKIELFVVPGGSVESASLHMARAVSRRLERRIKAASELTEINANVLLYANMLSNILFMHALISNKRLNIPEKIWSIHRVSLE

SEQ ID NO: 2 - protein, 172 aa, synthetic construct
  source 1..172: note = T33-51 protein cage, Subunit B
MRITTKVGDKGSTRLFGGEEVWKDDPIIEANGTLDELTSFIGEAKHYVDEEMKGILEEIQNDIYKIMGEIGSKGKIEGISEERIKWLAGLIERYSEMVNKLSFVLPGGTLESAKLDVCRTIARRAERKVATVLREFGIGTLAAIYLALLSRLLFLLARVIEIEKNKLKEVRS

SEQ ID NO: 3 - protein, 124 aa, synthetic construct
  source 1..124: note = T33-31 protein cage, Subunit A
MVRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQLE

SEQ ID NO: 4 - protein, 105 aa, synthetic construct
  source 1..105: note = T33-31 protein cage, Subunit B
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTGLE

SEQ ID NO: 5 - protein, 172 aa, synthetic construct
  source 1..172: note = T33-21 protein cage, Subunit A
MRITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 6 - protein, 125 aa, synthetic construct
  source 1..125: note = T33-21 protein cage, Subunit B
MPHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQRLE

SEQ ID NO: 7 - protein, 158 aa, synthetic construct
  source 1..158: note = T33-28 protein cage, Subunit A
MESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 8 - protein, 115 aa, synthetic construct
  source 1..115: note = T33-28 protein cage, Subunit B
MPVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNFLE

SEQ ID NO: 9 - protein, 205 aa, synthetic construct
  source 1..205: note = I53-50 protein cage, Subunit A
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 10 - protein, 167 aa, synthetic construct
  source 1..167: note = I53-50 protein cage, Subunit B
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAAGSLEHHHHHH

SEQ ID NO: 11 - protein, 114 aa, synthetic construct
  source 1..114: note = I53-47 protein cage, Subunit A
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 12 - protein, 167 aa, synthetic construct
  source 1..167: note = I53-47 protein cage, Subunit B
MNQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAAGSLEHHHHHH

SEQ ID NO: 13 - protein, 156 aa, synthetic construct
  source 1..156: note = I53-40 protein cage, Subunit A
MTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 14 - protein, 213 aa, synthetic construct
  source 1..213: note = I53-40 protein cage, Subunit B
MSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNPGSLE

SEQ ID NO: 15 - protein, 156 aa, synthetic construct
  source 1..156: note = I52-32 protein cage, Subunit A
MKYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFNLE

SEQ ID NO: 16 - protein, 138 aa, synthetic construct
  source 1..138: note = I52-32 protein cage, Subunit B
MGMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 17 - protein, 157 aa, synthetic construct
  source 1..157: note = I32-28 protein cage, Subunit A
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 18 - protein, 159 aa, synthetic construct
  source 1..159: note = I32-28 protein cage, Subunit B
MILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPALE

SEQ ID NO: 19 - protein, 183 aa, synthetic construct
  source 1..183: note = Human H chain Ferritin protein cage
MTTASTSQVRQNYHQDSEAAINRQINLELYASYVYLSMSYYFDRDDVALKNFAKYFLHQSHEEREHAEKLMKLQNQRGGRIFLQDIQKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 20 - protein, 318 aa, synthetic construct
  source 1..318: note = Sulfur Oxygenase Reductase (SOR) protein cage
MPKPYVAINMAELKNEPKTFEMFASVGPKVCMVTARHPGFVGFQNHIQIGILPFGNRYGGAKMDMTKESSTVRVLQYTFWKDWKDHEEMHRQNWSYLFRLCYSCASQMIWGPWEPIYEIIYANMPINTEMTDFTAVVGKKFAEGKPLDIPVISQPYGKRVVAFAEHSVIPGKEKQFEDAIVRTLEMLKKAPGFLGAMVLKEIGVSGIGSMQFGAKGFHQVLENPGSLEPDPNNVMYSVPEAKNTPQQYIVHVEWANTDALMFGMGRVLLYPELRQVHDEVLDTLVYGPYIRILNPMMEGTFWREYLNENAWRHPQFGG

SEQ ID NO: 21 - protein, 33 aa, synthetic construct
  source 1..33: note = DARPin repeat consensus sequence
  VARIANT 2..3: note = Each Xaa can independently be any amino acid
  VARIANT 5: note = Xaa can be any amino acid
  VARIANT 13..14: note = Each Xaa can independently be any amino acid
  VARIANT 20: note = Xaa can be any amino acid
  VARIANT 26: note = Xaa can be any amino acid
  VARIANT 33: note = Xaa can be any amino acid
DXXGXTPLHLAAXXGHLEIXEVLLKXGADVNAX

SEQ ID NO: 22 - protein, 33 aa, synthetic construct
  source 1..33: note = DARPin repeat sequence
DKDGYTPLHLAAREGHLEIVEVLLKAGADVNAK

SEQ ID NO: 23 - protein, 126 aa, synthetic construct
  source 1..126: note = HER2-ECD-specific DARPin
GSDSGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLN

SEQ ID NO: 24 - protein, 159 aa, synthetic construct
  source 1..159: note = AcrB-specific DARPin
GSDLGKKLLEAARAGQDDEVRILMANGADVNARDFTGWTPLHLAAHFGHLEIVEVLLKNGADVNAKDSLGVTPLHLAARRGHLEIVEVLLKNGADVNASDSHGFTPLHLAAKRGHLEIVEVLLKNGADVNAQDKFGKTAFDISIDNGNEDLAEILQKLN

SEQ ID NO: 25 - protein, 124 aa, synthetic construct
  source 1..124: note = Caspase-3-specific DARPin
DLGKKLLEATRAGQDDEVRILMANGADVNAMDDAGVTPLHLAAKRGHLEIVEVLLKHGADVNARDIWGRTPLHLAATVGHLEIVEVLLEYGADVNAQDKFGKTAFDISIDNGNEDLAEILQKLN

SEQ ID NO: 26 - protein, 159 aa, synthetic construct
  source 1..159: note = IL-13-specific DARPin
GSDLDKKLLEAARAGQDDEVRILMANGADVNARDSYGSTPLHLAAREGHLEIVEVLLKYGADVNAADFIGDTPLHLAAYRGHLEIVEVLLKYGADVNASDITGETPLHLAAQIGHLEIVEVLLKHGADVNAQDKFGKTPADIAADNGHEDIAEVLQKLN

SEQ ID NO: 27 - protein, 159 aa, synthetic construct
  source 1..159: note = MBP-specific DARPin
GSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 28 - protein, 156 aa, synthetic construct
  source 1..156: note = sfGFP-specific DARPin
QGKKLLEAARAGQDDEVRILMANGADVNAADDVGVTPLHLAAQRGHLEIVEVLLKCGADVNAADLWGQTPLHLAATAGHLEIVEVLLKNGADVNARDNIGHTPLHLAAWAGHLEIVEVLLKYGADVNAQDKFGKTPFDLAIDNGNEDIAEVLQKAA

SEQ ID NO: 29 - protein, 60 aa, synthetic construct
  source 1..60: note = Affibody consensus sequence
  VARIANT 9..11: note = Each Xaa can independently be any amino acid
  VARIANT 13..14: note = Each Xaa can independently be any amino acid
  VARIANT 17..18: note = Each Xaa can independently be any amino acid
  VARIANT 24..25: note = Each Xaa can independently be any amino acid
  VARIANT 27..28: note = Each Xaa can independently be any amino acid
  VARIANT 32: note = Xaa can be any amino acid
  VARIANT 35: note = Xaa can be any amino acid
  VARIANT 59..60: note = Each Xaa can independently be any amino acid
VDNKFNKEXXXAXXEIXXLPNLNXXQXXAFIXSLXDDPSQSANLLAEAKKLNDAQAPKXX

SEQ ID NO: 30 - protein, 58 aa, synthetic construct
  source 1..58: note = Affibody sequence
VDNKFNKELSVAGREIVTLPNLNDPQKKAFIFSLWDDPSQSANLLAEAKKLNDAQAPK

SEQ ID NO: 31 - protein, 58 aa, synthetic construct
  source 1..58: note = HER2 receptor-specific affibody
VDNKFNKEMRNAYWEIALLPNLNNQQKRAFIRSLYDDPSQSANLLAEAKKLNDAQAPK

SEQ ID NO: 32 - protein, 60 aa, synthetic construct
  source 1..60: note = Amyloid beta-specific affibody
VDNKFNKEMASAGGEIVYLPNLNPDQLCAFIHSLHDDPSQSANLLAEAKKLNDAQAPKVD

SEQ ID NO: 33 - protein, 58 aa, synthetic construct
  source 1..58: note = Protein A-specific affibody
VDNKFNKETQEASWEIFTLPNLNGRQVAAFISSLLDDPSQSANLLAEAKKLNDAQAPK

SEQ ID NO: 34 - protein, 473 aa, synthetic construct
  source 1..473: note = Exemplary LOC based on sulfur oxygenase protein cage
MPKPYVAINMAELKNEPKTFEMFASVGPKVCMVTARHPGFVGFQNHIQIGILPFGNRYGGAKMDMTKESSTVRVLQYTFWKDWKDHEEMHRQNWSYLFRLCYSCASQMIWGPWEPIYEIIYANMPINTEMTDFTAVVGKKFAEGKPLDIPVISQPYGKRVVAFAEHSVIPGKEKQFEDAIVRTLEMLKKAPGFLGAMVLKEIGVSGIGSMQFGAKGFHQVLENPGSLEPDPNNVMYSVPEAKNTPQQYIVHVEWANTDALMFGMGRVLLYPELRQVHDEVLDTLVYGPYIRILNPMMEGTFWREYLNEAAAQGKKLLEAARAGQDDEVRILMANGADVNAADDVGVTPLHLAAQRGHLEIVEVLLKCGADVNAADLWGQTPLHLAATAGHLEIVEVLLKNGADVNARDNIGHTPLHLAAWAGHLEIVEVLLKYGADVNAQDKFGKTPFDLAIDNGNEDIAEVLQKAAHHHHHH

SEQ ID NO: 35 - protein, 185 aa, synthetic construct
  source 1..185: note = Exemplary LOC based on T33-51 protein cage, Chain A
MFTRRGDQGETDLANRARVGKDSPVVEVQGTIDELNSFIGYALVLSRWDDIRNDLFRIQNDLFVLGEDVSTGGKGRTVTMDMIIYLIKRSVEMKAEIGKIELFVVPGGSVESASLHMARAVSRRLERRIKAASELTEINANVLLYANMLSNILFMHALISNKRLNIPEKIWSIHRVSLEHHHHHH

SEQ ID NO: 36 - protein, 328 aa, synthetic construct
  source 1..328: note = Exemplary LOC based on T33-51 protein cage, Chain B
MVTGYRLFEKESGSGSTRLFGGEEVWKDDPIIEANGTLDELTSFIGEAKHYVDEEMKGILEEIQNDIYKIMGEIGSKGKIEGISEERIKWLAGLIERYSEMVNKLSFVLPGGTLESAKLDVCRTIARRAERKVATVLREFGIGTLAAIYLALLSRLLFLLARVIEIEKNKLAQGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 37 - protein, 12 aa, synthetic construct
  source 1..12: note = Exemplary Passenger Molecule
MVTGYRLFEKES

SEQ ID NO: 38 - protein, 179 aa, synthetic construct
  source 1..179: note = T33-51 protein cage, Subunit A
  VARIANT 1..22: note = Each Xaa can independently be any amino acid
  VARIANT 172..179: note = Each Xaa can independently be any amino acid
XXXXXXXXXXXXXXXXXXXXXXSPVVEVQGTIDELNSFIGYALVLSRWDDIRNDLFRIQNDLFVLGEDVSTGGKGRTVTMDMIIYLIKRSVEMKAEIGKIELFVVPGGSVESASLHMARAVSRRLERRIKAASELTEINANVLLYANMLSNILFMHALISNKRLNIPEKIWXXXXXXXX

SEQ ID NO: 39 - protein, 172 aa, synthetic construct
  source 1..172: note = T33-51 protein cage, Subunit B
  VARIANT 1..23: note = Each Xaa can independently be any amino acid
  VARIANT 98..104: note = Each Xaa can independently be any amino acid
  VARIANT 168..172: note = Each Xaa can independently be any amino acid
XXXXXXXXXXXXXXXXXXXXXXXDDPIIEANGTLDELTSFIGEAKHYVDEEMKGILEEIQNDIYKIMGEIGSKGKIEGISEERIKWLAGLIERYSEMXXXXXXXLPGGTLESAKLDVCRTIARRAERKVATVLREFGIGTLAAIYLALLSRLLFLLARVIEIEKNKLXXXXX

SEQ ID NO: 40 - protein, 124 aa, synthetic construct
  source 1..124: note = T33-31 protein cage, Subunit A
  VARIANT 118..124: note = Each Xaa can independently be any amino acid
MVRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDXXXXXXX

SEQ ID NO: 41 - protein, 105 aa, synthetic construct
  source 1..105: note = T33-31 protein cage, Subunit B
  VARIANT 104..105: note = Each Xaa can independently be any amino acid
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTGXX

SEQ ID NO: 42 - protein, 172 aa, synthetic construct
  source 1..172: note = T33-21 protein cage, Subunit A
  VARIANT 1..22: note = Each Xaa can independently be any amino acid
  VARIANT 169..172: note = Each Xaa can independently be any amino acid
XXXXXXXXXXXXXXXXXXXXXXKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKXXXX

SEQ ID NO: 43 - protein, 125 aa, synthetic construct
  source 1..125: note = T33-21 protein cage, Subunit B
  VARIANT 1: note = Xaa can be any amino acid
  VARIANT 120..125: note = Each Xaa can independently be any amino acid
XPHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVXXXXXX

SEQ ID NO: 44 - protein, 158 aa, synthetic construct
  source 1..158: note = T33-28 protein cage, Subunit A
  VARIANT 1..2: note = Each Xaa can independently be any amino acid
  VARIANT 95..99: note = Each Xaa can independently be any amino acid
XXSVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVXXXXXEVERETAEEEAKLALRVALQVDIADEHSCVTQFEMKLREELLSSDSFHPDKDEYYKDFL

SEQ ID NO: 45 - protein, 115 aa, synthetic construct
  source 1..115: note = T33-28 protein cage, Subunit B
  VARIANT 114..115: note = Each Xaa can independently be any amino acid
MPVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNFXX

SEQ ID NO: 46 - protein, 205 aa, synthetic construct
  source 1..205: note = I53-50 protein cage, Subunit A
  VARIANT 204..205: note = Each Xaa can independently be any amino acid
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCXX

SEQ ID NO: 47 - protein, 167 aa, synthetic construct
  source 1..167: note = I53-50 protein cage, Subunit B
  VARIANT 1..10: note = Each Xaa can independently be any amino acid
  VARIANT 156..167: note = Each Xaa can independently be any amino acid
XXXXXXXXXXTVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIXXXXXXXXXXXX

SEQ ID NO: 48 - protein, 114 aa, synthetic construct; identical to SEQ ID NO: 11
  source 1..114: note = I53-47 protein cage, Subunit A

SEQ ID NO: 49 - protein, 167 aa, synthetic construct
  source 1..167: note = I53-47 protein cage, Subunit B
  VARIANT 1..10: note = Each Xaa can independently be any amino acid
  VARIANT 160..167: note = Each Xaa can independently be any amino acid
XXXXXXXXXXTVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAAGSXXXXXXXX

SEQ ID NO: 50 - protein, 156 aa, synthetic construct
  source 1..156: note = I53-40 protein cage, Subunit A
  VARIANT 1: note = Xaa can be any amino acid
  VARIANT 144..156: note = Each Xaa can independently be any amino acid
XTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGXXXXXXXXXXXXX

SEQ ID NO: 51 - protein, 213 aa, synthetic construct
  source 1..213: note = I53-40 protein cage, Subunit B
  VARIANT 1: note = Xaa can be any amino acid
  VARIANT 210..213: note = Each Xaa can independently be any amino acid
XSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNPXXXX

SEQ ID NO: 52 - protein, 156 aa, synthetic construct
  source 1..156: note = I52-32 protein cage, Subunit A
  VARIANT 84..92: note = Each Xaa can independently be any amino acid
  VARIANT 122..134: note = Each Xaa can independently be any amino acid
MKYDGSKLRIGILHARWNLEIIAALVAGAIKRLQEFGVKAENIIIETVPGSFELPYGSKLFVEKQKRLGKPLDAIIPIGVLIKXXXXXXXXXCDSTTHQLMKLNFELGIPVIFGVLTCLTDXXXXXXXXXXXXXMHNHGEDWGAAAVEMATKFNLE

SEQ ID NO: 53 - protein, 138 aa, synthetic construct
  source 1..138: note = I52-32 protein cage, Subunit B
  VARIANT 1..2: note = Each Xaa can independently be any amino acid
  VARIANT 134..138: note = Each Xaa can independently be any amino acid
XXMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKXXXXX

SEQ ID NO: 54 - protein, 157 aa, synthetic construct
  source 1..157: note = I32-28 protein cage, Subunit A
  VARIANT 153..157: note = Each Xaa can independently be any amino acid
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDXXXXX

SEQ ID NO: 55 - protein, 159 aa, synthetic construct
  source 1..159: note = I32-28 protein cage, Subunit B
  VARIANT 1..2: note = Each Xaa can independently be any amino acid
  VARIANT 117..123: note = Each Xaa can independently be any amino acid
  VARIANT 158..159: note = Each Xaa can independently be any amino acid
XXLSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATXXXXXXXGAAFEVMVQAAAGVTLLLVAMALPQGLAAGLPPAXX

SEQ ID NO: 56 - protein, 183 aa, synthetic construct
  source 1..183: note = Human H Subunit Ferritin protein cage
  VARIANT 1..5: note = Each Xaa can independently be any amino acid
  VARIANT 178..183: note = Each Xaa can independently be any amino acid
XXXXXTSQVRQNYHQDSEAAINRQINLELYASYVYLSMSYYFDRDDVALKNFAKYFLHQSHEEREHAEKLMKLQNQRGGRIFLQDIQKPDCDDWESGLNAMECALHLEKNVNQSLLELHKLATDKNDPHLCDFIETHYLNEQVKAIKELGDHVTNLRKMGAPESGLAEYLFDKHTLGXXXXXX

SEQ ID NO: 57 - protein, 327 aa, synthetic construct
  source 1..327: note = Exemplary LOC based on T33-51 protein cage, Chain B
MCGGGRITTKVGDKGSTRLFGGEEVWKDDPIIEANGTLDELTSFIGEAKHYVDEEMKGILEEIQNDIYKIMGEIGSKGKIEGISEERIKWLAGLIERYSEMNKLSFVLPGGTLESAKLDVCRTIARRAERKVATVLREFGIGTLAAIYLALLSRLLFLLARVIEIEKNKLAQGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQKLN

SEQ ID NO: 58 - protein, 14 aa, synthetic construct
  source 1..14: note = Exemplary Passenger Molecule
MCGGGRITTKVGDK

(19) United States
(12) Patent Application Publication
(10) Pub. No.: US 2025/0155439 A1
(43) Pub. Date: May 15, 2025

(54) Ligand Operable Protein Cages

(71) Applicant: The Regents of the University of California, Oakland, CA (US)

(72) Inventors: Todd Owen Yeates, Agoura Hills, CA (US); Justin Miller, Santa Fe, NM (US); Eric Jinsuk Lee, Los Angeles, CA (US)

(21) Appl. No.: 18/834,817

(22) PCT Filed: Feb. 8, 2023
(86) PCT No.: PCT/US2023/062186

§ 371 Date: Jul. 31, 2024

Related U.S. Application Data
(60) Provisional application No. 63/308,243, filed on Feb. 9, 2022.

(51) Int. Cl. G01N 33/573; C07K 14/00; C12N 9/02; G01N 33/542

(52) U.S. Cl. CPC G01N 33/573; C07K 14/001; C12N 9/0069; C12Y 113/11055; G01N 33/542; G01N 2333/90241

(57) ABSTRACT

Disclosed herein are protein cages that can be assembled and disassembled on command and methods and compositions thereof.

### CROSS-REFERENCE TO RELATED APPLICATIONS

This application claims the benefit of U.S. Patent Application No. 63/308,243, filed Feb. 9, 2022, which is herein incorporated by reference in its entirety.

### ACKNOWLEDGEMENT OF GOVERNMENT SUPPORT

This invention was made with Government support under DE-FC02-02ER63421 awarded by the U.S. Department of Energy. The Government has certain rights in the invention.

### REFERENCE TO A SEQUENCE LISTING SUBMITTED VIA EFS-WEB

The content of the ASCII text file of the sequence listing named “20230207_034044_234WO1_seq_ST26” which is 101,824 bytes in size was created on Feb. 7, 2023, and electronically submitted via Patent Center herewith the application, is incorporated herein by reference in its entirety.

### BACKGROUND OF THE INVENTION

### 1. Field of the Invention

The field of the invention generally relates to engineered self-assembling protein cages and their use as molecular diagnostics and drug delivery agents.

### 2. Description of the Related Art

Self-assembling protein cages are known in the art. See, for example, U.S. Pat. Nos. 8,969,521, 9,066,870, 9,630,994, 10,248,758, 10,501,733, US20200397886, US20210163540, and WO2020/220044. Self-assembling protein cages comprise a plurality of polypeptide subunits that are held together via intermolecular forces to form a protein shell having an interior cavity. While the prior art protein cages may be disassembled by exposing the cages to denaturing conditions, e.g., solvents, acids, bases, salts, and/or heat, such denaturing conditions are often unsuitable for applications under physiological conditions.

Thus, a need exists for self-assembling protein cages that are capable of being disassembled using denaturants that specifically target the self-assembling protein cages instead of denaturants (e.g., acids, bases, salts, solvents, etc.) and physical conditions (e.g., heat and mechanical agitation) that non-specifically denature proteins generally.

### SUMMARY OF THE INVENTION

In some embodiments, the present invention is directed to a ligand operable protein cage (LOC), which comprises a plurality of subunits bound together by non-covalent interactions, wherein at least one subunit comprises a sequence that has (a) at least 90% sequence identity over a comparison window of at least X-30 consecutive amino acid residues of a subunit protein having X number of amino acid residues, said subunit protein being of a known protein cage, and (b) a given binder covalently attached to the N-terminus or the C-terminus of the at least one subunit. In some embodiments, the known protein cage is selected from the group consisting of T33-51, T33-31, T33-21, T33-28, I53-40, I52-32, I32-28, ferritin, and sulfur oxygenase reductase. In some embodiments, the sequence of the subunit protein is selected from the group consisting of SEQ ID NOs: 1-20 and SEQ ID NOs: 38-56. In some embodiments, X is an integer selected from the range of about 90 to about 225, preferably about 105 to about 213. In some embodiments, the comparison window is at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of the subunit protein. In some embodiments, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, over the length of the comparison window, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, the sequence of the subunit protein is SEQ ID NO: 2 or SEQ ID NO: 34. In some embodiments, the given binder is an scFv, a Fab fragment, a Fab′ fragment, a F(ab′)₂ fragment, an sdFv, an Fv, a protein comprising a set of CDRs of an antibody, a camelid nanobody, an affibody, an affilin, an affimer, an affitin, an alphabody, an anticalin, an avimer, a DARPin, an Fynomer, a Kunitz domain peptide, a monobody, or a nanoCLAMP known in the art. In some embodiments, the given binder is a DARPin or an affibody. In some embodiments, the DARPin comprises SEQ ID NO: 21. In some embodiments, the affibody comprises SEQ ID NO: 29. In some embodiments, the non-covalent interactions between the subunit and one of its adjacent subunits are disrupted when the given binder has its cognate ligand bound thereto. In some embodiments, a passenger molecule of interest is covalently attached to the at least one subunit. In some embodiments, the LOC comprises a passively packaged passenger molecule of interest is in its interior cavity. In some embodiments, the passenger molecule is a detectable label (e.g., a fluorophore), a detection reagent, a therapeutic agent of interest, a theradiagnostic of interest, or a radiotheranostic of interest. In some embodiments, the passively packaged passenger molecule is a detectable label (e.g., a fluorophore), a detection reagent, a therapeutic agent of interest, a theradiagnostic of interest, or a radiotheranostic of interest.

In some embodiments, the present invention is directed to a method of delivering or releasing a passenger molecule of interest and/or a passively packaged passenger molecule of interest via a ligand operable protein cage (LOC), which comprises a plurality of subunits bound together by non-covalent interactions, wherein at least one subunit comprises a sequence that has (a) at least 90% sequence identity over a comparison window of at least X-30 consecutive amino acid residues of a subunit protein having X number of amino acid residues, said subunit protein being of a known protein cage, and (b) a given binder covalently attached to the N-terminus or the C-terminus of the at least one subunit, wherein said method comprises contacting the given binder with its cognate ligand. In some embodiments, the known protein cage is selected from the group consisting of T33-51, T33-31, T33-21, T33-28, I53-40, I52-32, I32-28, ferritin, and sulfur oxygenase reductase. In some embodiments, the sequence of the subunit protein is selected from the group consisting of SEQ ID NOs: 1-20 and SEQ ID NOs: 38-56. In some embodiments, X is an integer selected from the range of about 90 to about 225, preferably about 105 to about 213. In some embodiments, the comparison window is at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of the subunit protein. In some embodiments, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, over the length of the comparison window, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, the sequence of the subunit protein is SEQ ID NO: 2 or SEQ ID NO: 34. In some embodiments, the given binder is an scFv, a Fab fragment, a Fab′ fragment, a F(ab′)₂ fragment, an sdFv, an Fv, a protein comprising a set of CDRs of an antibody, a camelid nanobody, an affibody, an affilin, an affimer, an affitin, an alphabody, an anticalin, an avimer, a DARPin, an Fynomer, a Kunitz domain peptide, a monobody, or a nanoCLAMP known in the art. In some embodiments, the given binder is a DARPin or an affibody. In some embodiments, the DARPin comprises SEQ ID NO: 21. In some embodiments, the affibody comprises SEQ ID NO: 29. In some embodiments, the non-covalent interactions between the subunit and one of its adjacent subunits are disrupted when the given binder has its cognate ligand bound thereto. In some embodiments, a passenger molecule of interest is covalently attached to the at least one subunit. In some embodiments, the LOC comprises a passively packaged passenger molecule of interest is in its interior cavity. In some embodiments, the passenger molecule is a detectable label (e.g., a fluorophore), a detection reagent, a therapeutic agent of interest, a theradiagnostic of interest, or a radiotheranostic of interest. In some embodiments, the passively packaged passenger molecule is a detectable label (e.g., a fluorophore), a detection reagent, a therapeutic agent of interest, a theradiagnostic of interest, or a radiotheranostic of interest.

In some embodiments, the present invention is directed to an assay method which comprises using a ligand operable protein cage (LOC) to detect the presence or absence of the cognate ligand of a given binder in a sample by contacting the LOC with the sample and detecting the presence of absence of the cognate ligand bound to the given binder, wherein disassembly of the at least one subunit from the plurality of subunits indicates the presence of the cognate ligand. In some embodiments, the LOC comprises a plurality of subunits bound together by non-covalent interactions, wherein at least one subunit comprises a sequence that has (a) at least 90% sequence identity over a comparison window of at least X-30 consecutive amino acid residues of a subunit protein having X number of amino acid residues, said subunit protein being of a known protein cage, and (b) a given binder covalently attached to the N-terminus or the C-terminus of the at least one subunit. In some embodiments, the known protein cage is selected from the group consisting of T33-51, T33-31, T33-21, T33-28, I53-40, I52-32, I32-28, ferritin, and sulfur oxygenase reductase. In some embodiments, the sequence of the subunit protein is selected from the group consisting of SEQ ID NOs: 1-20 and SEQ ID NOs: 38-56. In some embodiments, X is an integer selected from the range of about 90 to about 225, preferably about 105 to about 213. In some embodiments, the comparison window is at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of the subunit protein. In some embodiments, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, over the length of the comparison window, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, the sequence of the subunit protein is SEQ ID NO: 2 or SEQ ID NO: 34. In some embodiments, the given binder is an scFv, a Fab fragment, a Fab′ fragment, a F(ab′)₂ fragment, an sdFv, an Fv, a protein comprising a set of CDRs of an antibody, a camelid nanobody, an affibody, an affilin, an affimer, an affitin, an alphabody, an anticalin, an avimer, a DARPin, an Fynomer, a Kunitz domain peptide, a monobody, or a nanoCLAMP known in the art. In some embodiments, the given binder is a DARPin or an affibody. In some embodiments, the DARPin comprises SEQ ID NO: 21. In some embodiments, the affibody comprises SEQ ID NO: 29. In some embodiments, the non-covalent interactions between the subunit and one of its adjacent subunits are disrupted when the given binder has its cognate ligand bound thereto. In some embodiments, a passenger molecule of interest is covalently attached to the at least one subunit. In some embodiments, the LOC comprises a passively packaged passenger molecule of interest is in its interior cavity. In some embodiments, the passenger molecule is a detectable label (e.g., a fluorophore), a detection reagent, a therapeutic agent of interest, a theradiagnostic of interest, or a radiotheranostic of interest. In some embodiments, the passively packaged passenger molecule is a detectable label, a detection reagent, a therapeutic agent of interest, a theradiagnostic of interest, or a radiotheranostic of interest.

In some embodiments, the present invention is directed to a composition or a kit which comprises one or more ligand operable protein cage (LOC), which comprises a plurality of subunits bound together by non-covalent interactions, wherein at least one subunit comprises a sequence that has (a) at least 90% sequence identity over a comparison window of at least X-30 consecutive amino acid residues of a subunit protein having X number of amino acid residues, said subunit protein being of a known protein cage, and (b) a given binder covalently attached to the N-terminus or the C-terminus of the at least one subunit. In some embodiments, the known protein cage is selected from the group consisting of T33-51, T33-31, T33-21, T33-28, I53-40, I52-32, I32-28, ferritin, and sulfur oxygenase reductase. In some embodiments, the sequence of the subunit protein is selected from the group consisting of SEQ ID NOs: 1-20 and SEQ ID NOs: 38-56. In some embodiments, X is an integer selected from the range of about 90 to about 225, preferably about 105 to about 213. In some embodiments, the comparison window is at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of the subunit protein. In some embodiments, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, over the length of the comparison window, the sequence of the at least one subunit has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, the sequence of the subunit protein is SEQ ID NO: 2 or SEQ ID NO: 34. In some embodiments, the given binder is an scFv, a Fab fragment, a Fab′ fragment, a F(ab′)₂ fragment, an sdFv, an Fv, a protein comprising a set of CDRs of an antibody, a camelid nanobody, an affibody, an affilin, an affimer, an affitin, an alphabody, an anticalin, an avimer, a DARPin, an Fynomer, a Kunitz domain peptide, a monobody, or a nanoCLAMP known in the art. In some embodiments, the given binder is a DARPin or an affibody. In some embodiments, the DARPin comprises SEQ ID NO: 21. In some embodiments, the affibody comprises SEQ ID NO: 29. In some embodiments, the non-covalent interactions between the subunit and one of its adjacent subunits are disrupted when the given binder has its cognate ligand bound thereto. In some embodiments, a passenger molecule of interest is covalently attached to the at least one subunit. In some embodiments, the LOC comprises a passively packaged passenger molecule of interest is in its interior cavity. In some embodiments, the passenger molecule is a detectable label (e.g., a fluorophore), a detection reagent, a therapeutic agent of interest, a theradiagnostic of interest, or a radiotheranostic of interest. In some embodiments, the passively packaged passenger molecule is a detectable label, a detection reagent, a therapeutic agent of interest, a theradiagnostic of interest, or a radiotheranostic of interest. In some embodiments, the kit further comprises the cognate ligand of the given binder. In some embodiments, the kit further comprises a reagent for detecting the disassembly of the at least one subunit from the plurality of subunits.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1: Schematically shows the components of LOCs, opening of the protein cage via the binder-ligand complex, and LOC variations.

FIG. 2: Modeling of the natural SO (sulfur oxidase) cage (tan color) fused to DARPin (light blue) that binds sfGFP. The binding domains of the DARPin are arranged to cause steric collision that disrupts the non-covalent interactions between subunits when sfGFP (green) is bound.

FIG. 3: Overlay of size exclusion chromatography curves showing destruction of cage assembly by addition of target ligand. Disruption of the LOC (natural sulfur oxygenase protein cage fused to a DARPin that binds sfGFP) by addition of sfGFP. SEC curves in the presence of sfGFP (dashed) and absence of sfGFP (dotted) show loss of the assembled form of the cage (indicated by shaded peak 1 on the chromatogram).

FIG. 4: Schematically illustrates the use of an LOC and a split-luciferase as a reporter molecule for use in diagnostic assays. The passenger molecule comprises the fragment of the luciferase enzyme that results in an observable signal.

FIG. 5-FIG. 8: Data showing the production and testing of a LOC (based on a designed protein cage) that signals the presence of MBP as the given ligand, on the basis of a split-luciferase complementation readout. FIG. 5: SDS following nickel affinity purification, confirming construction of the LOC from two protein subunits (cage subunit A modified by fusion to a split luciferase tag at the N-terminus and a binder (i.e., a DARPin that binds MBP) at the C-terminus, and cage subunit B). FIG. 6: SEC showing an early-eluting peak established to be the intended 24-subunit assembly. FIG. 7: Negative stain transmission electron microscopy confirming assembly of the cubic cages. FIG. 8: Luminescence readout demonstrating selectivity for disruption by the intended given ligand, MBP.

FIG. 9: Schematically illustrates a reporter system comprising an LOC and a self-quenching fluorophore as the passenger molecule. The fluorescent signal of the fluorophore is auto-quenched by a plurality of the same fluorophore molecule in close proximity (e.g., in the interior cavity of the LOC). When the LOC is “unlocked” via a ligand binding thereto, the LOC opens and thereby results in separation between the fluorophore molecules. The fluorescence of the fluorophore molecules increases as the space between the fluorophore molecules increases.

FIG. 10: Data demonstrating the production of a reporter system comprising an LOC and a fluorophore (i.e., Oregon Green-maleimide, a self-quenching fluorophore) as the passenger molecule. The fluorophore was covalently bound to an inward facing amino acid residue of a subunit of the LOC. The LOC was unlocked by its ligand, MBP, binding thereto. Lysozyme was used as a negative control ligand.

### DETAILED DESCRIPTION OF THE INVENTION

Disclosed herein are “Ligand Operable (Protein) Cages” (LOCs). As disclosed herein, LOCs are self-assembling protein cages that are engineered to unlock with a molecular key. Specifically, the LOCs have recombinantly fused to the outer surface of their protein cages one or more binders that bind a given ligand of interest. Upon binding the given ligand, the LOCs open or disassemble as a result of steric collision and/or steric interference caused by the intermolecular forces that hold the subunits of the protein cages together and thereby expose the interior cavity of the protein cages and any contents therein.

LOCs comprise two basic components—a protein cage and a binder recombinantly fused thereto.

### Protein Cages

As used herein, a “protein cage” refers to a plurality of polypeptides (“subunits” or “oligomeric protein units”) which collectively form a three-dimensional structure having an outer surface and interior cavity. The amino acid sequences of the subunits may be the same or different. Typically, protein cages are formed with (a) subunits having the same amino acid sequence, or (b) 2-3 different of subunits, e.g., some subunits have a first amino acid sequence and some subunits have a second amino acid sequence. Each subunit of a protein cage typically occurs in multiple copies (e.g., 12, 24, or 60 copies), and at least one of the subunits has one or more amino acids at or near a terminal end that is exposed on the exterior surface of the protein cage.

Generally, the subunits of the protein cages assemble into a symmetric geometric shapes that mimic the 3D shape of any one of the Platonic solids: tetrahedron, cube, octahedron, icosahedron, and dodecahedron. Typically, non-covalent interactions that do not involve the sharing of electrons (e.g., electrostatic interactions, Van der Waals forces, π-effects, hydrophobic effects, etc.) hold the subunits together, thereby forming the protein cage. The non-covalent interactions are generally stable under most physiological conditions. The protein cages may be disassembled or opened to expose their interior cavities by disrupting the non-covalent interactions, e.g., applying competing thermodynamic forces. In some embodiments, the protein cages are composed of 12 pentameric oligomeric protein units; 6 tetrameric oligomeric protein units; 4, 8, or 20 trimeric oligomeric protein units; 6, 12, or 30 dimeric protein units; or a combination thereof, which are held together by non-covalent interactions.

Protein cages known in the art may be modified to be an LOC according to the present invention. That is, a prior art protein cage may be modified, using methods in the art, to have a binder recombinantly fused to one or more of its oligomeric protein units. Exemplary protein cages include: T33-51, T33-31, T33-21, T33-28, I53-40, I52-32, I32-28, ferritin, and sulfur oxygenase reductase, which are composed of subunits having the following amino acid sequences:

T33-51 protein cage:
Subunit A:
(SEQ ID NO: 1)
mftrrgdqgetdlanrarvgkdspvvevqgtidelnsfigyalvLSRWDdiRNdlFRiqNdlfVl
gEdvsTgGKGRTVTmDmiiYlikRsvEmkAEigKIELfvvpGGSVEsaslhmaravsrrlerrik
aaselteinanvllyanmlsnilfmhalisnkRLNIPEKIWSIHRVSLE
Subunit B:
(SEQ ID NO: 2)
mrittkvgdkgstrlfggeevwkddpiieangtldeltsfigeakhYVDEEmkGilEEiqNDiyK
imGeigsKGKIEGISeEriKWlaGliERyseMVNKLSFVLPGGTLesakldvcrtiarraerkva
tvlrefgigtlaaiylallsrllfllarvieIEKNKLKEVRS
T33-31 protein cage:
Subunit A:
(SEQ ID NO: 3)
MVrgirgaitveedtpaailaatiellLKmlEANGIQSYEElaaviftvtedltsafpaeaARLI
GMHRvpllsarevpvpgslprvirvlalwNTDTPQDRVRHVYLNEaVRlRPDLESAQLE
Subunit B:
(SEQ ID NO: 4)
MEevvlitvpsaLvavKiahAlveERLAAcvnivpgltsiyrEEGSVvsdhellllvktTTDafP
KlkervkElHPYEVpeivaLPIAEGnReylDwlREntgle
T33-21 protein cage:
Subunit A:
(SEQ ID NO: 5)
mrittkvgdkgstrlfggeevwkdspiieangtldeltsfigeaKHYVDEEmkGilEEiqNdiYK
imGeigskGKIEGISEEriaWllKlilRymEMVNLKSFVLPGGTLEsakldvcrtiarralrkvl
tvtrefgigaeaaaYllalsdllfllarvieIEKNKLKEVRS
Subunit B:
(SEQ ID NO: 6)
mphlvieatanLRLETSPGEllEqanKalFasgqfGEAdiksrfVTLEAYRQGTAAVERaylhac
lsildgrdiatrtllgaslcavlaEAVAGGGEEGvqvsvevremerlsyakrvvargrle
T33-28 protein cage:
Subunit A:
(SEQ ID NO: 7)
mesvntsflspslvtirdfdngqfavlRIGRTGfpadkgdidlcldkmigvraagiflgddtedg
fkgphirircVDIDDKHTYNAMvyvdlIVGTGASEveretaeeeaklalrvalqvdiaDEHSCVT
QFEMKLREELLSSDSFHpdkdEyyKDFL
Subunit B:
(SEQ ID NO: 8)
MPviqtfvstpldhhkrlllaiiyrivtrvVLGKPEDLvmmtfhdstpmhffgsTDPvacvrvea
LGGYGPSepEkvtSivtAaiTavcgiVADrifvlyfSPLHCGWNGINFLE
153-50 protein cage:
Subunit A:
(SEQ ID NO: 9)
MKMEElFkkhkiVavlransveeaiekavavfaggvhlieitftvpdadtvikalsvlkEkGaii
gagtvtsVeqcRkavEsGAEfivsphldEeisQfckEKGvfympgvmtptelvkamkLGHTilkl
fpgevvgpqfVkamkgpfpnvkfvptggvnldnvcewfkagvlavgvgsalvkgtpdevrekaka
fvekirgcte
Subunit B:
(SEQ ID NO: 10)
MNQHSHKDYETVriavvrarwhAeivDacvSafEaamAdigGDRFAVDVEDVPGayeiplhartl
aeTGRygavlgtafvvnggiyrhefvasavidgmmnvqlstGvpvlsavltphryRDSdahtllf
lalfavkgmeaaracvEilAaREKIAAGSLEHHHHHH
153-47 protein cage:
Subunit A:
(SEQ ID NO: 11)
mpiftlntNIKATDVPSDflsltsrlvglilskpgsyvavhintDQQLSFGGSTNpaafgtlmsi
ggiepsknrdhsavlfdhlnaMLGIPKNRmyihfvnlngddvgwngttf
Subunit B:
(SEQ ID NO: 12)
MNQHSHKDYETVriavvrarwhAdivDacvEafEiamAaigGDRFAVDVfdvpgayeiplhartl
aeTGRygavlgtafvvnggiyrhefvasavidgmmnvqlstGvpvlsavltphryrdsaehhrff
aahfavkgveaaraciEilAaREKIAAGSLEHHHHHH
153-40 protein cage:
Subunit A:
(SEQ ID NO: 13)
mtkkvgivdttfarvdmasaailtlkmespnikiirktvpgikdlpvackklleeegcdivmalg
mpGKAEkdkvcaheaslglmlaqlmtnkhiievfvheDEAKDDaelkilaarraiehalnvyyll
fkpeyltrmagkglrggfEDAGPARE
Subunit B:
(SEQ ID NO: 14)
mstinNqlkalkvipviaidnaediiPlgKvlaEnglpaaeitfrssaavkaimllrsaqpemli
gagtilngvqalaakeagatfvvspgfnpntvracqiigidivpgvnnpstveaalemglttlkf
fpaeASGGISmvkSlvgpygdirlmptgGiTPSNIdNyLAIPqvlacggtWMVdkKLvtNGEwdE
iaRltrEiveQVNPGSLE
152-32 protein cage:
Subunit A:
(SEQ ID NO: 15)
MKYDGSKLrigilharwnleiiaalvagaikrlqeFGVKAENiiietvpgsfelpygsklfvekq
KRLGKPLDaiipigvlikgstmhfeyicdstthqlmklnfELGIpvifgvltcltdeqaearagl
iegkmhnhgedwgaaaveMATKENLE
Subunit B:
(SEQ ID NO: 16)
MGMKEkfvliithgdfgkgllsgaeviigkqenvhtvglnlgdniekvakevmriiiaklaedke
iiivvDlFGGspfNiaLemMKTFDVKvitginmPmlvEllTsinVYdtTEllEnisKigkDGIKV
IEKSSLKM
132-28 protein cage:
Subunit A:
(SEQ ID NO: 17)
MGDDariaaigdvdelnsqigvllaeplpddvraalsAiqHdlfDlgGelCIPGHAAITEdhllr
lalwlvhyngqlppleefilpggargaalahvertvcrraersikalgaSEPLNIapaayvnlls
dllfvlarvinrAaggadvlwdrtrah
Subunit B:
(SEQ ID NO: 18)
MILSAEQSFTLRHPHGQAAaLAfvRepaAalAGVQRLRGLDSDGEqvwgellvrvpllgevdlpf
rseIVRTPQGAelrpLTLTGERAWVAVSGQATAAEGGEMAFAFQFQAHlatpeaegeggaafevm
vqaaagvtlllvamalpqglaAGLPPALE
Human H chain Ferritin protein cage:
(SEQ ID NO: 19)
mttastsqvrqnyhqDseAainRqinLelYasyvylsmsyyfdrddvalknfakyflhqsheere
haeklmklqnqrggRIFLQDIQKPDCDDWEsglNameCalHlekNvnQsllElhkLatDKndphl
cdfiethylneqvkaikelgdhvTnlRKMGApESglaeylfdkhtlgdsdnes
Sulfur Oxygenase Reductase (SOR) protein cage:
(SEQ ID NO: 20)
MPKPYvainmaelknepktfemfasvgpkvcmvtaRHPGfvgfqnhiqigilpfgnryggakmd
mtkesstvrvlqytfwKDWKDheEmhRqnwsylfrlcyscasqmiwgpwepIYEIIYAnmpin
temtdftavvgkkfaEGKPldipvisqpygkrvvafaehsvipgKeKqfeDaiVrtlEmlkkap
gflgamvlkeigvsgigsmqfgakgfhqvlenpgslepdpNnvMYSvpEakntpqqyivhvewa
ntdalmfgmgrvllypElrqVhdEvldtlvygpyirilnpmmegtfwrEYLNENAWRHPQFGG

In the above sequences, the capital letters represent “exterior residues”, i.e., amino acid residues that are exposed on the exterior surface of the protein cages. The bold underlined font in the above sequences indicates regions that are more amenable to amino acid substitutions, additions, and deletions compared to the non-bolded regions. Thus, the bold underlined amino acid residues may be each independently Xaa, thereby making SEQ ID NOs: 1-19, SEQ ID NOs: 38-56, respectively. In some embodiments, a LOC having a subunit that corresponds to all or part of a sequence selected from the group consisting of SEQ ID NOs: 1-20 and SEQ ID NOs: 38-56 comprises a binder that is covalently attached to an exterior residue or replaces one or more exterior residues, preferably within 20 amino acid residues from the N-terminus or C-terminus. Likewise, a passenger molecule that is to be carried on the exterior surface of a LOC is preferably covalently attached to an exterior residue or replaces one or more exterior residues, which exterior residues need not be at or near one of the terminal ends. Conversely, a passenger molecule that is to be carried by covalent attachment to the interior surface of a LOC is preferably covalently attached to a non-exterior residue or replaces one or more non-exterior residues. For example, a binder may be covalently attached to one of the first 12 residues of SEQ ID NO: 12 or provided in place of one or more of the first 12 residues of SEQ ID NO: 12. Alternatively, a binder may be covalently attached to one of the last 16 residues of SEQ ID NO: 12 or provided in place of one or more of the last 16 residues of SEQ ID NO: 12. In embodiments where a binder and a passenger molecule are provided on the same subunit, preferably the binder and the passenger molecule are provided at or near opposite terminal ends of the subunit amino acid chain. For example, the binder is located at or near the C-terminus and the passenger molecule is located at or near the N-terminus of the same amino acid chain forming the given subunit.

As exemplified in the detailed Examples, the subunits of the exemplary LOCs that have a binder recombinantly fused thereto are the subunits of protein cages known in the art that are truncated by 5 or 10 amino acids at their C-terminal end. Example 2 exemplifies an embodiment where 10 amino acids at the N-terminus of the subunit known in the art is substituted with a passenger molecule. Thus, in some embodiments, the up to about 20, up to about 19, up to about 18, up to about 17, up to about 16, up to about 15, up to about 14, up to about 13, up to about 12, up to about 11, up to about 10, up to about 9, up to about 8, up to about 7, up to about 6, up to about 5, up to about 4, up to about 3, up to about 2, or up to about 1 amino acid residues at the N-terminus or C-terminus of a subunit of a protein cage known in the art is replaced with a binder and optionally a passenger molecule at the opposite terminal end.

Therefore, in some embodiments, a subunit of a LOC corresponds to a comparison window of at least X-30, at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of a subunit sequence of a protein cage known in the art, wherein “X” is the number of amino acids in the subunit sequence. In some embodiments, X is an integer selected from the range of about 90 to about 225, preferably about 105 to about 213. In some embodiments, a subunit of an LOC corresponds to a comparison window of at least X-30, at least X-29, at least X-28, at least X-27, at least X-26, at least X-25, at least X-24, at least X-23, at least X-22, at least X-21, at least X-20, at least X-19, at least X-18, at least X-17, at least X-16, at least X-15, at least X-14, at least X-13, at least X-12, at least X-11, at least X-10, at least X-9, at least X-8, at least X-7, at least X-6, at least X-5, at least X-4, at least X-3, at least X-2, at least X-1, or X number of consecutive amino acids of a sequence selected from the group consisting of SEQ ID NOs: 1-20 and SEQ ID NOs: 38-56.

In some embodiments, over the length of the comparison window, the sequence of the subunit of the LOC has at least 90%, at least 91%, at least 92%, at least 93%, at least 94%, at least 95%, at least 96%, at least 97%, at least 98%, at least 99%, or 100% sequence identity to a sequence selected from the group consisting of SEQ ID NOs: 1-20 and SEQ ID NOs: 38-56. In some embodiments, a LOC has a sequence that has at least 90%, at least 91%, at least 92%, at least 93%, at least 94%, at least 95%, at least 96%, at least 97%, at least 98%, at least 99%, or 100% sequence identity to the full-length a sequence selected from the group consisting of SEQ ID NOs: 1-20 and SEQ ID NOs: 38-56.

Thus, for example, if the subunit sequence of a protein cage known in the art is 100 amino acids long, a subunit of a LOC corresponds to a comparison window of “at least X-30” means that the comparison window is at least 70 amino acids in long and 90% sequence identity over the comparison window means that of the 70 amino acids at least 63 amino acids in the comparison window must be identical whereas 90% sequence identity over the full-length means that the subunit sequence must comprise, at a minimum, 90 amino acids that are identical over the comparison window because 90% of 100 is 90 amino acid residues. If, however, the comparison window is, e.g., at least X-5, then the comparison window is at least 95 amino acids long and a sequence identity of at least 90% over the comparison window means that at least 86 amino acids in the comparison window must be identical whereas 90% sequence identity over the full-length means that the subunit sequence may differ by up to 5 amino acid residues.

### Binders

As used herein, a “binder” refers to a protein that specifically binds a given ligand of interest. Suitable binders include single chain Fvs (scFvs), Fab fragments, Fab′ fragments, F(ab′)₂, disulfide linked Fvs (sdFvs), Fvs, fragments comprising or consisting of the CDRs of the VH and/or VL chain of an antibody, camelid nanobodies, and “antibody mimetics” such as affibodies, affilins, affimers, affitins, alphabodies, anticalins, avimers, DARPins, Fynomers, Kunitz domain peptides, monobodies, and nanoCLAMPs.

The binder itself, when fused to a subunit, does not disrupt the non-covalent interactions between subunits of a protein cage. Thus, in some embodiments, the binder is about 5-50 kDa with a length or width of about 25-50 Å. However, the binder-ligand complex, i.e., the binder bound to its ligand, sterically shifts the amino acids of the subunit and/or an adjacent subunit and thereby disrupts, i.e., breaks the non-covalent interactions between the subunit and the adjacent subunit. That is, the ligand when bound to the binder sterically disrupts the intermolecular forces that hold the subunits together such that the protein cage unlocks, i.e., opens and/or disassembles. As used herein, a protein cage that is “open” means that the interior cavity (and any contents therein) of the protein cage is exposed to the exterior. Each subunit of a protein cage need not be completely separated from its adjacent subunit to be an “opened” protein cage. Instead, an “opened” protein cage may comprise some subunits that are still non-covalently attached to each other. That is, some of the non-covalent interactions between the subunits of the protein cage may remain intact. For example, an “opened” protein cage may have an open slit between two subunits; the slit being where the non-covalent interactions between two adjacent subunits have been disrupted. As another example, an “opened” protein cage may comprise two halves of the protein cage, which halves are completely separated from each other.

Short, rigid linkers are generally less tolerant than flexible linkers with changes in steric effects. As such, short, rigid linkers are preferred over flexible linkers for covalently attaching a binder to a subunit. In some embodiments, the linker between a binder and its subunit is 10, 9, 8, 7, 6, 5, 4, 3, 2, or 1 amino acids long. In some embodiments, the linker is 1-5 amino acids long. In some embodiments, the linker is 6-10 amino acids long. In some embodiments, the secondary structure of the linker is an alpha helical structure comprising up to 10 amino acids.

In some embodiments, the binder is “modular” which means that the amino acids that bind a given ligand may be readily substituted (e.g., using recombinant methods in the art) with different amino acids to cause the binder to bind a different ligand. That is, the binder may comprise a scaffold that presents a binding domain, whereby the amino acids in the binding domain may be readily substituted. For example, the binder may be an affibody scaffold in which the variable target binding residues are readily substituted. As another example, the binder may be a DARPin wherein the amino acids in the loop regions are readily substituted.

In some embodiments, the binder is a DARPin. As used herein, a “DARPin” refers to a protein having a repetitive structure, usually 3 or more repeats of a unit (usually about 33 amino acids long comprising a beta turn, two antiparallel alpha helices, and a loop), with the amino acid sequence of the repeating unit based on the natural protein ankyrin and its homologues. In some embodiments, a DARPin comprises an N- and C-cap, and two or more repeats having binding domains that are capable of binding its given cognate ligand. DARPin sequences vary across different proteins and organisms where they are found, but show recognizable sequence similarity, especially in the helical (non-loop regions). An example consensus sequence for a DARPin repeat is:

(SEQ ID NO: 21)
DXXGXTPLHLAAXXGHLEIXEVLLKXGADVNAX

In some embodiments, the DARPin repeat is:

(SEQ ID NO: 22)
DKDGYTPLHLAAREGHLEIVEVLLKAGADVNAK

The following are examples of DARPins which have a DARPin repeat that falls within the scope of SEQ ID NO: 21:

HER2-ECD-specific DARPin, SEQ ID NO: 23:
GSDSGKKLLEAARAGQDDEVRILMANGADVNAKDEYGLTPLYLAT
AHGHLEIVEVLLKNGADVNAVDAIGFTPLHLAAFIGHLEIAEVLL
KHGADVNAQDKEGKTAFDISIGNGNEDLAEILQKLN
AcrB-specific DARPin, SEQ ID NO: 24:
GSDLGKKLLEAARAGQDDEVRILMANGADVNARDFTGWTPLHLAA
HFGHLEIVEVLLKNGADVNAKDSLGVTPLHLAARRGHLEIVEVLL
KNGADVNASDSHGFTPLHLAAKRGHLEIVEVLLKNGADVNAQDKF
GKTAFDISIDNGNEDLAEILQKLN
Caspase-3-specific DARPin, SEQ ID NO: 25:
DLGKKLLEATRAGQDDEVRILMANGADVNAMDDAGVTPLHLAAKR
GHLEIVEVLLKHGADVNARDIWGRTPLHLAATVGHLEIVEVLLEY
GADVNAQDKEGKTAFDISIDNGNEDLAEILQKLN
IL-13-specific DARPin, SEQ ID NO: 26:
GSDLDKKLLEAARAGQDDEVRILMANGADVNARDSYGSTPLHLAA
REGHLEIVEVLLKYGADVNAADFIGDTPLHLAAYRGHLEIVEVLL
KYGADVNASDITGETPLHLAAQIGHLEIVEVLLKHGADVNAQDKF
GKTPADIAADNGHEDIAEVLQKLN
MBP-specific DARPin, SEQ ID NO: 27:
GSDLGRKLLEAARAGQDDEVRILMANGADVNAADNTGTTPLHLAA
YSGHLEIVEVLLKHGADVDASDVFGYTPLHLAAYWGHLEIVEVLL
KNGADVNAMDSDGMTPLHLAAKWGYLEIVEVLLKHGADVNAQDKF
GKTAFDISIDNGNEDLAEILQKLN
sfGFP-specific DARPin, SEQ ID NO: 28:
QGKKLLEAARAGQDDEVRILMANGADVNAADDVGVTPLHLAAQRG
HLEIVEVLLKCGADVNAADLWGQTPLHLAATAGHLEIVEVLLKNG
ADVNARDNIGHTPLHLAAWAGHLEIVEVLLKYGADVNAQDKFGKT
PFDLAIDNGNEDIAEVLQKAA

Therefore, in some embodiments, the binder is a DARPin that has a DARPin repeat sequence that comprises, consists of, or consists essentially of SEQ ID NO: 21. In some embodiments, the binder is a DARPin that has a DARPin repeat sequence that comprises, consists of, or consists essentially of a sequence that has at least 90%, at least 91%, at least 92%, at least 93%, at least 94%, at least 95%, at least 96%, at least 97%, at least 98%, or at least 99% sequence identity to SEQ ID NO: 22. In some embodiments, the DARPin has at least 90%, at least 91%, at least 92%, at least 93%, at least 94%, at least 95%, at least 96%, at least 97%, at least 98%, at least 99%, or 100% sequence identity to SEQ ID NO: 23, SEQ ID NO: 24, SEQ ID NO: 25, SEQ ID NO: 26, SEQ ID NO: 27, or SEQ ID NO: 28.

The molecular weight of the DARPins are about 13-26 kDa and have the following approximate dimensions: 45×30×20 Å (Her2-ECD), 55×30×20 Å (AcrB), 40×30×20 Å (Caspase 3), 55×30×20 Å (IL-13), and 45×30×20 Å (MBP). Thus, in some embodiments, the binder is a DARPin having a molecular weight of about 10-30 kDa, about 10-26 kDa, about 10-20 kDa, about 11-19 kDa, about 12-18 kDa, or about 13-17 kDa and/or a width and height of about 15-35 Å, preferably about 20-30 Å, and a length ranging from about 35-65 Å, preferably about 40-60 Å.

In some embodiments, the binder is an affibody. An example consensus affibody sequence is:

(SEQ ID NO: 29)
VDNKFNKEXXXAXXEIXXLPNLNXXQXXAF
IXSLXDDPSQSANLLAEAKKLNDAQAPKXX,

wherein one or both Xaa at the C-terminus may be present or absent).

The following are examples of affibodies that fall within the scope of SEQ ID NO: 29:

SEQ ID NO: 30:
VDNKENKELSVAGREIVTLPNLNDPQKKA
FIFSLWDDPSQSANLLAEAKKLNDAQAPK
HER2 receptor-specific affibody, SEQ ID NO: 31:
VDNKENKEMRNAYWEIALLPNLNNQQKRA
FIRSLYDDPSQSANLLAEAKKLNDAQAPK
Amyloid beta-specific affibody, SEQ ID NO: 32:
VDNKENKEMASAGGEIVYLPNLNPDQLCAF
IHSLHDDPSQSANLLAEAKKLNDAQAPKVD
Protein A-specific affibody, SEQ ID NO: 33:
VDNKENKETQEASWEIFTLPNLNGRQVAAF
ISSLLDDPSQSANLLAEAKKLNDAQAPK

Therefore, in some embodiments, the affibody comprises, consists of, or consists essentially of a sequence having at least 90%, at least 91%, at least 92%, at least 93%, at least 94%, at least 95%, at least 96%, at least 97%, at least 98%, at least 99%, or 100% sequence identity to SEQ ID NO: 30, SEQ ID NO: 31, SEQ ID NO: 32, or SEQ ID NO: 33.

The molecular weight of the affibodies above are about 6-7 kDa and have the following approximate dimensions: about 15×20×35 Å. Thus, in some embodiments, the binder is an affibody having a molecular weight of about 6-7 kDa and/or a length, width, or height ranging from about 10-40 Å, preferably about 15-35 Å.

### Passenger Molecule

The LOCs may optionally comprise one or more passenger molecules. As used herein, a “passenger molecule” refers to a molecule of interest that is carried on the surface of or within the internal cavity of a LOC. The one or more passenger molecules may be covalently or non-covalently linked to the outward facing surface of the protein cage. In some embodiments, the one or more passenger molecules may be a freely soluble molecule that is contained within the internal cavity of the protein cage that is released to the exterior when the protein cage is opened or disassembled. For example, the one or more passenger molecules may be passively packaged within the cavity of the protein cage by assembling the protein cage in a solution comprising the one or more passenger molecules. In some embodiments, the one or more passenger molecules may be covalently or non-covalently attached to the interior surface of the protein cage that defines the internal cavity. For example, the one or more passenger molecules may be recombinantly fused to the N-terminus of one or more subunits forming the protein cage. Passenger molecules may be a chemical or a biomolecule. Passenger molecules may be naturally occurring or synthetic. Passenger molecules may be therapeutic agents (e.g., a drug) or diagnostic agents (e.g., a detectable label such as a fluorophore). The one or more passenger molecules of a given LOC may be the same or different. In some embodiments, the one or more passenger molecules is a detectable label such as a fluorophore. In some embodiments, the fluorophore is a self-quenching fluorophore.

Exemplary therapeutic agents include antineoplastic agents such as doxorubicin, cisplatin, carboplatin, paclitaxel, gemcitabine; nucleic acid molecules such as DNA and RNA including siRNAs and miRNAs; antibiotics; enzymatic inhibitors such as DPP-IV and prolyl endopeptidase inhibitors, angiotensin-I converting enzyme inhibitors; amyloid inhibitors; antivirals; bioactive peptides such as insulin and peptide hormones; melittin; antimicrobials; bovine hemoglobin α-chain fragments; porcine myofibrillar fragments; cell receptor agonists and antagonists; theradiagnostics; and radiotheranostics such as radionuclides (e.g., alpha and beta emitters).

Exemplary diagnostic agents include luminescent and fluorescent labels; X-ray, MRI and PET imaging agents such as Ga-68, fluorodeoxyglucose-18, and radioactive isotopes (including beta and gamma emitters); detectable nanoparticles such as iron oxide and gold nanoparticles; and organic dyes. In some embodiments, the detectable label is a reporter molecule, e.g., a molecule that becomes detectable in the presence of another molecule.

In some embodiments, the one or more passenger molecules is a cell penetrating polypeptide (CPP) such as the TAT peptide from HIV, Penetratin, Transportan, and Xentry.

In some embodiments, an LOC can operate in a reverse, or reversible, fashion. That is, a ligand whose presence causes the LOC to open may be removed or inactivated, thereby leading to reassembly of the LOC. For example, the ligand may be physically removed or inactivated by, e.g., chemical, biochemical, or thermal degradation, or by competitive binding. In the latter case, a displacing biomolecule may bind to the ligand more tightly than the ligand binds to the LOC. If the ligand can only bind to one of those—the displacing biomolecule or the LOC—in a mutually exclusive fashion, then the presence of the displacing biomolecule will lead to LOC (re) assembly, even in the presence of the ligand. LOC systems where reassembly of the cage might be readily monitored include especially those where the LOC signal is based either on encapsulation of covalently attached FRET passenger molecules, or on covalently attached split proteins. With in vitro diagnostic applications using those systems, LOC disruption might be fully, or nearly fully, reversed by ligand removal, thereby leading to decreased fluorescence from the passenger molecule acting as the FRET donor.

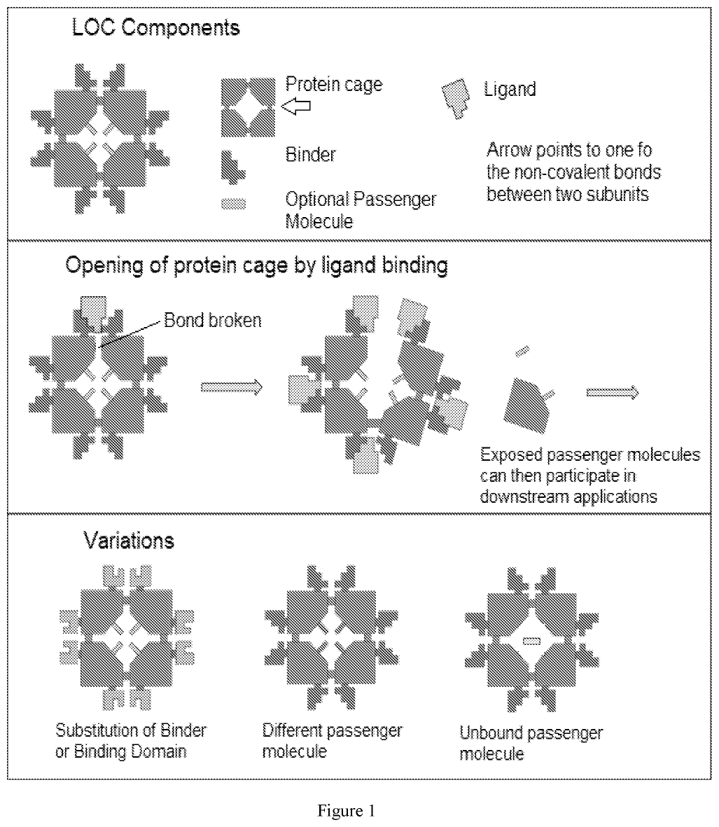

FIG. 1 schematically shows the components of LOCs, the non-covalent interactions between subunits being disrupted by the steric effects caused by the binder-ligand complex which thereby results in the protein cage opening, exposed passenger molecules that are consequently allowed to participate in downstream applications, e.g., as diagnostic or therapeutic agents, and LOC variations in which the binder or the binding domain of the binder is substituted, a different passenger molecule is provided, and the passenger molecule is not covalently linked to the interior surface of the protein cage that defines the internal cavity.

### Methods of Designing LOCs

Prior art protein cages displaying binders, e.g., DARPins, are generally designed so that the protein cages remain stable, i.e., intact, where the binders bind their target ligands. Such prior art protein cages often employ flexible linkers to link the binders to the subunits because the flexible linkers help avoid steric collisions and steric interference. Contrary to the prior art, LOCs are intentionally designed so that the binder-ligand complex is sterically and thermodynamically incompatible with the cage subunits remaining in their assembled form. In other words, the protein cage with its protruding binders without their ligands being bound thereto is stable, but the formation of the binder-ligand complex results in steric collision and repulsion that thereby disrupt the non-covalent interactions between the subunits of the protein cage.

Thus, LOCs are designed so that steric clashes are unavoidable when the binder binds its target ligand and the steric clashes then disrupt the non-covalent interactions between the subunits. In some embodiments, the binder is covalently attached to the surface of the protein cage so that the ligand of interest almost, but not fully, fits the binding domain of the binder. Because the protein cage is itself held together by non-covalent interactions, the thermodynamic forces for binding between the binder and the ligand prevail and drive the dissociation and disruption of the protein cage, according to well-established principles of mass action. Variations on the steric-exclusion principle are possible, e.g., the LOC can be designed so that a single copy of the given ligand can bind to one of the protruding binders, while binding of additional ligands to their adjacent cognate binders are sterically forbidden.

In some embodiments, the linker that is used to attach a binder to a subunit of a given LOC is a rigid linker so as to prevent, inhibit, or minimize spatial flexibility of the position of the binder. In some embodiments, the linker is about up to about 10, up to about 9, up to about 8, up to about 7, up to about 6, up to about 5, up to about 4, up to about 3, up to about 2, or 1 amino acid(s) in length. In some embodiments, the linker is an alpha-helical protein structure known in the art.

### Downstream Applications

LOCs may be used for a variety of therapeutic and diagnostic/imaging applications. For example, an LOC having a therapeutic agent, e.g., a chemotherapeutic, as a passenger molecule contained within its internal cavity, and a binder that specifically binds a given tumor marker may be used for targeted drug delivery. In such embodiments, upon formation of the binder-ligand complex, the protein cage opens or disassembles and thereby releases the chemotherapeutic directly at the site of cells expressing the tumor marker. Such drug delivery may be targeted to any cell of interest by selecting a binder that specifically binds a ligand that is characteristic of the cell of interest. In embodiments where one of the passenger molecules is a cell-penetrating polypeptide, entry of a therapeutic agent may be facilitated.

As another example, an LOC may have a binder that specifically binds a ligand that is expressed by a cell in the presence of a given toxin and a passenger molecule in its internal cavity, which passenger molecule neutralizes the given toxin. Thus, the LOC opens upon binding the toxin and thereby releases the neutralizing passenger molecule at the site of the toxin. Alternatively, an LOC may have a binder that specifically binds a ligand which is an epitope of a toxigenic microorganism, e.g., Enterotoxigenic Escherichia coli (ETEC), and upon binding the LOC opens and releases the passenger molecule, which may be therapeutic, e.g., an antibacterial that prevents or inhibits the growth of the microorganism, an anti-toxin that neutralizes the toxin that is released by the microorganism, a chemokine that induces an immune response against the microorganism, etc.

The premise of using an LOC as a diagnostic or imaging agent is similar. For example, an LOC having a passenger molecule contained within its internal cavity that cannot be detected within an intact LOC but becomes a detectable label when exposed to the environment exterior to the internal cavity, and a binder that specifically binds a target ligand of interest. In such embodiments, the formation of the binder-ligand complex causes the protein cage to open or disassemble and thereby releases the passenger molecule which is then a detectable label, which indicates the presence of the target ligand of interest. For example, the passenger molecules may be self-quenching fluorophores and when the protein cage opens or disassembles, the fluorophores separate from each other and their fluorescence becomes detectable. As another example, the LOC may have a binder that specifically binds a given tumor marker. In such embodiments, the formation of the binder-ligand complex causes the protein cage to open or disassemble and thereby releases the passenger molecule which is then a detectable label and thereby indicates the presence or location of cancer cells expressing the given tumor marker.

The proteins discussed herein may be made using methods known in the art including chemical synthesis, biosynthesis (in vitro and in vivo synthesis) using recombinant DNA methods, and solid phase synthesis. See, e.g., Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. The proteins may be purified using protein purification techniques known in the art such as reverse phase high-performance liquid chromatography (HPLC), ion-exchange or immunoaffinity chromatography, filtration or size exclusion, or electrophoresis. See, e.g., Olsnes and Pihl (1973) Biochem 12 (16): 3121-3126; and Scopes (1982) Protein Purification, Springer-Verlag, NY, which are herein incorporated by reference. Alternatively, the proteins may be made by recombinant DNA techniques known in the art. Thus, polynucleotides that encode a protein cage subunit fused to a binder (a “cage-binder fusion”) are contemplated herein.

In some embodiments, the LOCs and/or cage-binder fusions are substantially purified. As used herein, a “substantially purified” compound refers to a compound that is removed from its natural environment and/or is at least about 60% free, preferably about 75% free, and more preferably about 90% free, and most preferably about 95-100% free from other macromolecular components or compounds with which the compound is associated with in nature or from its synthesis.

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)₂, disulfide linked Fvs (sdFvs), camelid nanobodies, Fvs, and fragments comprising or alternatively consisting of, either a VL or a VH domain.

As used herein, a compound (e.g., receptor or antibody) “specifically binds” a given target (e.g., ligand or epitope) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e.g., a given ligand or epitope). An example of an antibody that specifically binds a given target is an antibody that binds the given target with greater affinity, avidity, more readily, and/or with greater duration than it does to other compounds. An antibody that specifically binds a given target over a specified alternative is an antibody that binds the given target with greater affinity, avidity, more readily, and/or with greater duration than it does to the specified alternative.

As used herein, “binding affinity” refers to the propensity of a compound to associate with (or alternatively dissociate from) a given target and may be expressed in terms of its dissociation constant, Kd. In some embodiments, the binders have a Kd of 10⁻⁵ M or less, 10⁻⁶ M or less, preferably 10⁻⁷ M or less, more preferably 10⁻⁸ M or less, even more preferably 10⁻⁹ M or less, and most preferably 10⁻¹⁰ M or less, to their given target. Binding affinity can be determined using methods in the art, such as equilibrium dialysis, equilibrium binding, gel filtration, immunoassays, surface plasmon resonance, biolayer interferometry and spectroscopy using experimental conditions that exemplify the conditions under which the compound and the given target may come into contact and/or interact. Dissociation constants may be used to determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of a binder for its cognate target is at least 1-fold or more, preferably at least 5-fold or more, more preferably at least 10-fold or more, and most preferably at least 100-fold or more than its binding affinity for the specified alternative.

As used herein, the term “sample” is used in its broadest sense and includes specimens and cultures obtained from any source, as well as biological samples and environmental samples. Biological samples may be obtained from animals (including humans) and encompass fluids, solids, tissues, and gases. Biological samples include blood products, such as plasma, serum, and the like. A biological sample can be obtained from a subject using methods in the art. A sample to be analyzed using one or more methods described herein can be either an initial unprocessed sample taken from a subject or a subsequently processed, e.g., partially purified, diluted, concentrated, fluidized, pretreated with a reagent (e.g., protease inhibitor, anti-coagulant, etc.), and the like. In some embodiments, the sample is a blood sample. In some embodiments, the blood sample is a whole blood sample, a serum sample, or a plasma sample. In some embodiments, the sample may be processed, e.g., condensed, diluted, partially purified, and the like. In some embodiments, the sample is pretreated with a reagent, e.g., a protease inhibitor. In some embodiments, two or more samples are collected at different time intervals to assess any difference in the amount of the analyte of interest, the progression of a disease or disorder, or the efficacy of a treatment. The test sample is then contacted with a capture reagent and, if the analyte is present, a conjugate between the analyte and the capture reagent is formed and is detected and/or measured with a detection reagent.

As used herein, a “capture reagent” refers to a molecule which specifically binds an analyte of interest. The capture reagent may be immobilized on a assay substrate. For example, if the analyte of interest is an antibody, the capture reagent may be an antigen or an epitope thereof to which the antibody specifically binds.

As used herein, an “assay substrate” refers to any substrate that may be used to immobilize a capture reagent thereon and then detect an analyte when bound thereto. Examples of assay substrates include membranes, beads, slides, and multi-well plates.

As used herein, a “detection reagent” refers to a substance that has a detectable label attached thereto and specifically binds an analyte of interest or a conjugate of the analyte of interest, e.g., an antibody-analyte conjugate.

As used herein, a “detectable label” is a compound or composition that produces or can be induced to produce a signal that is detectable by, e.g., visual, spectroscopic, photochemical, biochemical, immunochemical, or chemical means. The use of the term “labeled” as a modifier of a given substance, e.g., a labeled antibody, means that the substance has a detectable label attached thereto. A detectable label can be attached directly or indirectly by way of a linker (e.g., an amino acid linker or a chemical moiety). Examples of detectable labels include radioactive and non-radioactive isotopes (e.g., 125I, 18F, 13C, etc.), enzymes (e.g., β-galactosidase, peroxidase, etc.) and fragments thereof, enzyme substrates, enzyme inhibitors, coenzymes, catalysts, fluorophores (e.g., rhodamine, fluorescein isothiocyanate, etc.), dyes, chemiluminescers and luminescers (e.g., dioxetanes, luciferin, etc.), and sensitizers. A substance, e.g., antibody, having a detectable label means that a detectable label that is not linked, conjugated, or covalently attached to the substance, in its naturally-occurring form, has been linked, conjugated, or covalently attached to the substance by the hand of man. As used herein, the phrase “by the hand of man” means that a person or an object under the direction of a person (e.g., a robot or a machine operated or programmed by a person), not nature itself, has performed the specified act. Thus, the steps set forth in the claims are performed by the hand of man, e.g., a person or an object under the direction of the person.

### Immunoassays

In some embodiments, the present invention provides assays for detecting a given ligand. Such assays include any immunoassay format in the art such as enzyme immune assays (EIAs), magnetic immunoassays (MIAs), counting immunoassays (CIAs), chemiluminescent immunoassays (CLIAs), radioimmunoassays (RIAs), electrochemiluminescence immunoassays (ECLIA), fluorescent immunoassays (FIA), enzyme-linked immunosorbent assays (ELISAs), Western blot assays, and lateral flow tests (LFTs), and the like. The assays may be automated or manual. The various assays may employ any suitable labeling and detection system. The sensitivity and specificity of the assays can be further improved by optimizing the assay conditions, e.g., reaction times and temperatures, and/or modifying or substituting the reagents, e.g., different detection and labeling system, using methods in the art. In some embodiments, the immunoassay is an ELISA assay. In some embodiments, the immunoassay is a sandwich ELISA assay. In some embodiments, the immunoassay is a lateral flow assay.

### Kits

In some embodiments, the present invention provides kits for assaying a ligand of interest in a sample, e.g., a biological sample from a subject. In some embodiments, the kits comprise an LOC that is both (a) a capture reagent that, via the binder fused to the protein cage subunit, specifically binds the ligand, and (b) a detection reagent, i.e., a label as a passenger molecule contained within the internal cavity of the LOC that becomes a detectable label when the LOC opens and/or becomes disassembled. In some embodiments, the kits comprise an assay substrate for performing an immunoassay and immobilizing the capture reagent thereto. In some embodiments, the kits comprise one or more reagents, e.g., blocking buffers, assay buffers, diluents, wash solutions, etc., for performing the assay. In some embodiments, the kits comprise additional components such as interpretive information, control samples, reference levels, and standards.

In some embodiments, the present invention provides kits comprising one or more LOCs comprising one or more therapeutic agents as their passenger molecules, optionally in a composition, packaged together with one or more reagents or drug delivery devices. In some embodiments, the kits comprise the one or more LOCs, optionally in one or more unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Compositions

Compositions, including pharmaceutical compositions, comprising one or more LOCs are contemplated herein. The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more LOCs may be administered, preferably in the form of pharmaceutical compositions, to a subject. Preferably the subject is mammalian, more preferably, the subject is human. Preferred pharmaceutical compositions are those comprising at least one LOC in a therapeutically effective amount and a pharmaceutically acceptable vehicle. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the one or more LOC calculated to produce the desired therapeutic effect in association with the required pharmaceutically acceptable carrier. The specification for the dosage unit forms of the invention are dictated by and directly dependent on the unique characteristics of the given LOC and desired therapeutic effect to be achieved, and the limitations inherent in the art of compounding such an active compound for the treatment of individuals.

Toxicity and therapeutic efficacy of LOCs according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. LOCs which exhibit large therapeutic indices are preferred. While LOCs that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more LOCs can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### EXAMPLES

### Example 1: Sulfur Oxygenase as Exemplary Cage with a Binder for sfGFP

A natural protein cage, sulfur oxygenase (SO), was used to design an LOC. SO is a naturally self-assembling protein cage that is a 24-subunit homomeric structure. SO advantageously has alpha-helical C-termini exposed on its external surface. A DARPin having loop sequences that bind sfGFP (super-folder green fluorescent protein) was used as the binder. The C-termini from different cage subunits are, advantageously, in relatively close proximity, with closest approach of approximately 32 Å. Therefore, when designing the fusion of the DARPin to the cage subunit, the stereochemistry of the attachment was found suitable; modeling a continuous alpha helical connection between the cage subunit and the DARPin indicated a favorable positioning (i.e., partial steric occlusion) of the binding sites for the given ligand. Specifically, when modeled in three-dimensions, each binder (DARPin) was arranged such that the binding pocket faced towards the core of the LOC and a neighboring binder (DARPin). This positioning allowed for spacing of approximately 25 Å at closest approach. The bound sfGFP (the cognate ligand in this case) extends approximately 28 Å from the DARPin binder, thereby introducing a steric collision between cognate ligand and LOC.

The sequence of the exemplified LOC (SEQ ID NO: 34) is:

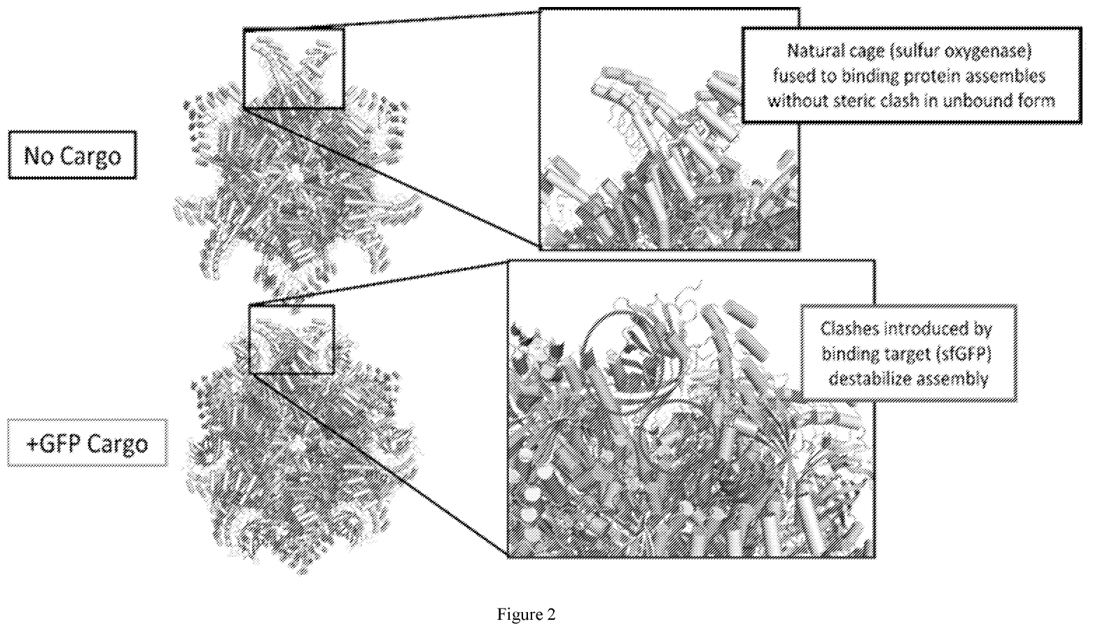

MPKPYVAINMAELKNEPKTFEMFASVGPKVCMVTARHPGFVGFQNH
IQIGILPEGNRYGGAKMDMTKESSTVRVLQYTFWKDWKDHEEMHR
QNWSYLFRLCYSCASQMIWGPWEPIYEIIYANMPINTEMTDFTAV
VGKKFAEGKPLDIPVISQPYGKRVVAFAEHSVIPGKEKQFEDAIV
RTLEMLKKAPGELGAMVLKEIGVSGIGSMQFGAKGFHQVLENPGS
LEPDPNNVMYSVPEAKNTPQQYIVHVEWANTDALMFGMGRVLLYP
ELRQVHDEVLDTLVYGPYIRILNPMMEGTFWREYLNEAAAQGKKL
LEAARAGQDDEVRILMANGADVNAADDVGVTPLHLAAQRGHLEIV
EVLLKCGADVNAADLWGQTPLHLAATAGHLEIVEVLLKNGADVNA
RDNIGHTPLHLAAWAGHLEIVEVLLKYGADVNAQDKFGKTPEDLA
IDNGNEDIAEVLQKAAHHHHHH

wherein the amino acid sequence of the natural SO (sulfur oxidase) cage (italics, SEQ ID NO: 20) with a 10 amino acid C-terminal truncation is fused via a linker (bold) to the sfGFP-binding DARPin (underlined, SEQ ID NO: 28). Protein models of this LOC and its bound ligand are shown in FIG. 2.

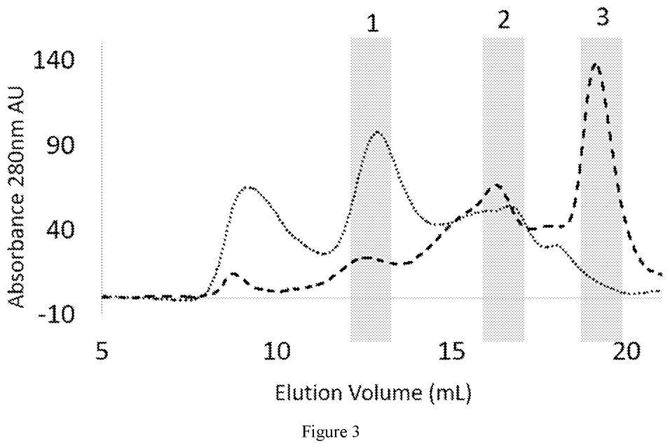

The size exclusion chromatography (SEC) experiments summarized in FIG. 3 demonstrate that the addition of the binder's cognate ligand (sfGFP) disrupts the three-dimensional structure of the protein cage of the LOC.

### Example 2: T33-51 as Exemplary Cage with a Binder for MBP and a Passenger Molecule

This experiment evidences that a passenger molecule contained in the interior cavity of an LOC is exposed to or released into the exterior environment. In this experiment, the LOC was expressed and purified, and then tested in a luminescence (light production) assay using the commercial nanoLuc version of the split luciferase enzyme.

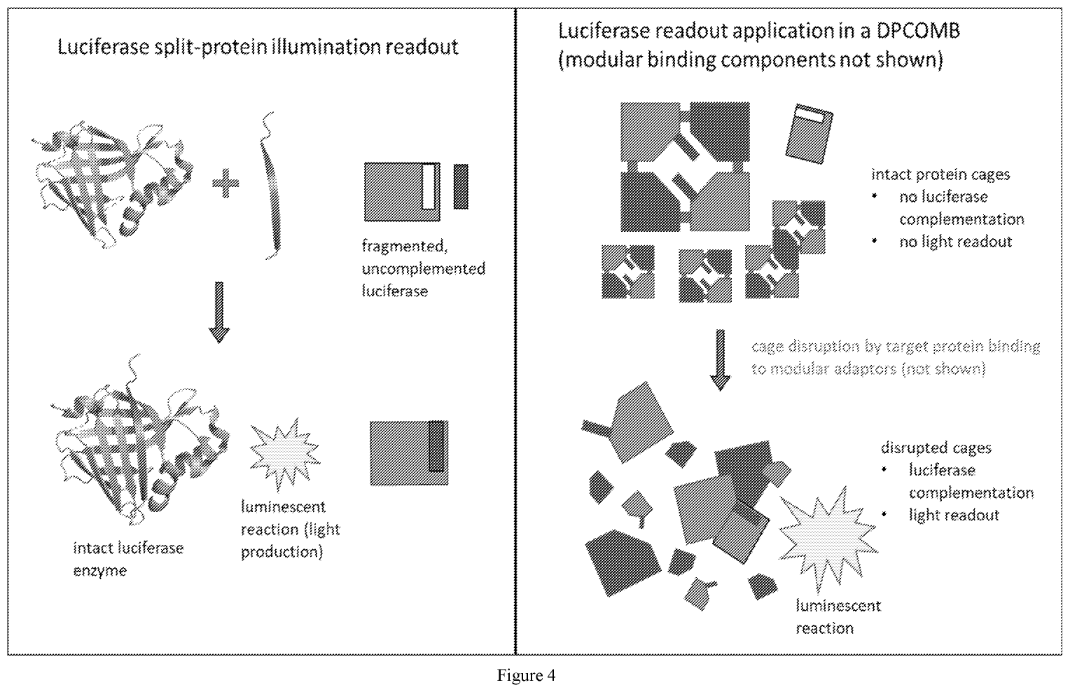

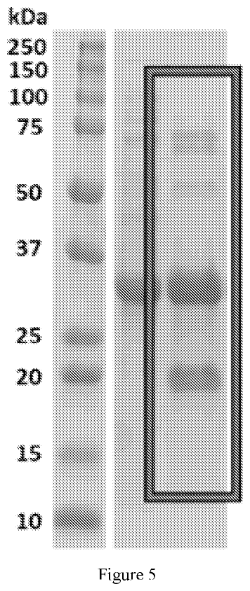

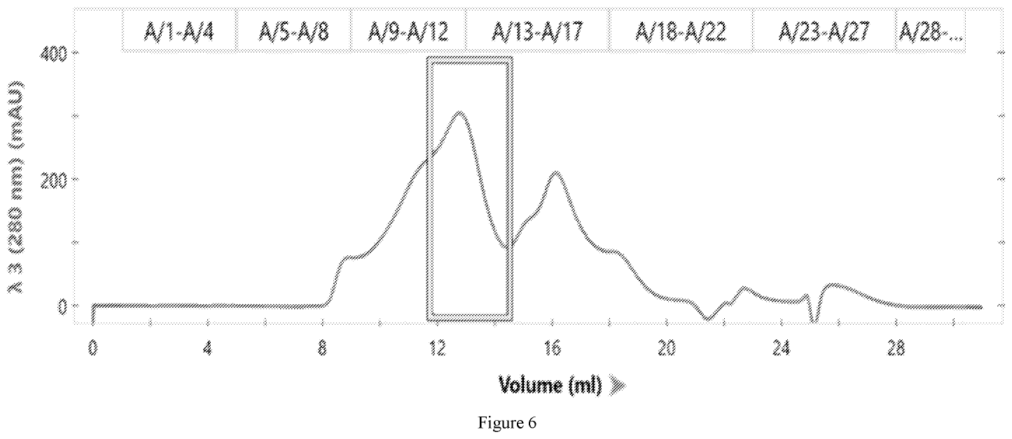

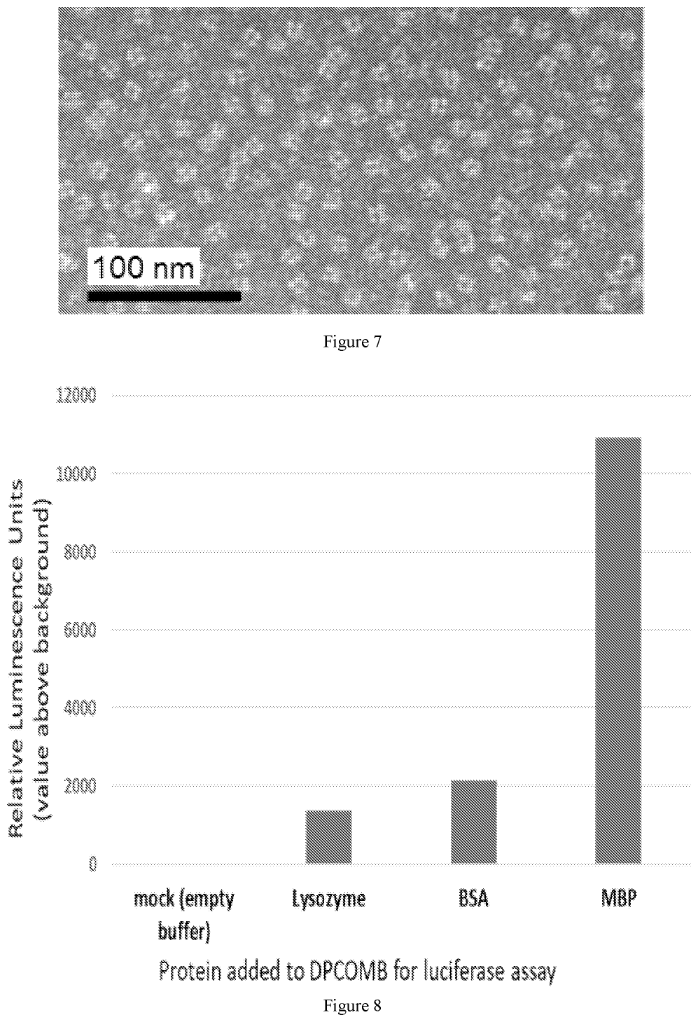

Here, the protein cage is T33-51, which is a 24 subunit protein cage shaped roughly like a cube. The binder is a DARPin with loop sequences that bind maltose binding protein (MBP). The passenger molecule is a small fragment of the split-luciferase enzyme. Upon opening or disassembly of the protein cage, the passenger molecule will be exposed to the larger fragment of the split-luciferase enzyme. Interaction between the two fragments results in a detectable label, i.e., a luciferase signal. This interaction and result are schematically illustrated FIG. 4 (which for simplicity, the binders are not shown).

The sequences of this LOC are as follows:

Chain A (SEQ ID NO: 35):
MFTRRGDQGETDLANRARVGKDSPVVEVQGTIDELNSFIGYALVL
SRWDDIRNDLFRIQNDLEVLGEDVSTGGKGRTVTMDMIIYLIKRS
VEMKAEIGKIELFVVPGGSVESASLHMARAVSRRLERRIKAASEL
TEINANVLLYANMLSNILFMHALISNKRLNIPEKIWSIHRVSLEH
HHHHH
Chain B (SEQ ID NO: 36):
MVTGYRLFEKESGSGSTRLEGGEEVWKDDPIIEANGTLDELTSFI
GEAKHYVDEEMKGILEEIQNDIYKIMGEIGSKGKIEGISEERIKW
LAGLIERYSEMVNKLSFVLPGGTLESAKLDVCRTIARRAERKVAT
VLREFGIGTLAAIYLALLSRLLELLARVIEIEKNKLAQ[GRKLLE
AARAGQDDEVRILMANGADVNAADNTGTTPLHLAAYSGHLEIVE
VLLKHGADVDASDVEGYTPLHLAAYWGHLEIVEVLLKNGADVNAM
DSDGMTPLHLAAKWGYLEIVEVLLKHGADVNAQDKFGKTAFDISI
DNGNEDLAEILQKLN]

The protein cage of T33-51 comprises two different subunits, i.e., Chains A and B (each present in 12 copies in the A12B12 assembly). The N-terminus of Chain A faces to the interior of the protein cage and the C-terminus of Chain A is exposed on the exterior of the protein cage. In the sequences above, Subunit A of T33-51 (SEQ ID NO: 1) is underlined; the portion of Subunit B of T33-51 is double underlined (i.e., SEQ ID NO: 2 with a 5 amino acid truncation at the C-terminus and a 10 amino acid truncation at the N-terminus); the passenger molecule (SEQ ID NO: 37) is italicized; the binder (MBP-specific DARPin, SEQ ID NO: 27, with a 4 amino acid N-terminal truncation) is bracketed; and the linker is in bold.

Luminescence was measured after addition of the cognate ligand, MBP. Control experiments were performed using two decoy proteins, lysozyme and bovine serum albumin. The data shown in FIG. 8 evidences that the protein cage is opened or disassembled and thereby exposes the passenger molecule contained within the interior cavity to the exterior upon the formation of the binder-ligand complex.

### Example 3: T33-51 as Exemplary Cage with a Binder for MBP and Self-Quenching Fluorophores as Passenger Molecules

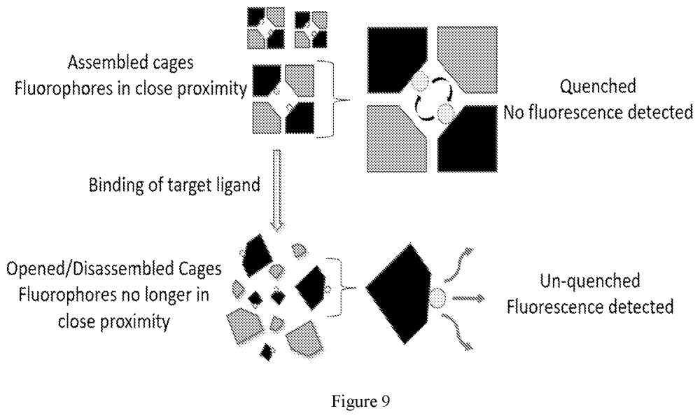

This experiment exemplifies the use of a plurality of fluorophores as passenger molecules packaged within the interior cavity of an LOC as a reporter system. As schematically shown in FIG. 9, when packaged together within the interior cavity, the fluorescent signal is low as a result of the self-quenching behavior of fluorophores; and when the LOC is opened via binding to its target ligand, the fluorescence of the fluorophores increases as a result of the fluorophores being separated from one another.

Here, the LOC is based on the T33-51 protein cage and the binder is a DARPin with loop sequences that bind maltose binding protein (MBP). Chain A is SEQ ID NO: 35 and the sequence of Chain B is:

(SEQ ID NO: 57)
MCGGGRITTKVGDKGSTRLEGGEEVWKDDPIIEANGTLDELTSFI
GEAKHYVDEEMKGILEEIQNDIYKIMGEIGSKGKIEGISEERIKW
LAGLIERYSEMNKLSFVLPGGTLESAKLDVCRTIARRAERKVATV
LREFGIGTLAAIYLALLSRLLELLARVIEIEKNKLAQ[GRKLLEA
ARAGQDDEVRILMANGADVNAADNTGTTPLHLAAYSGHLEIVEVL
LKHGADVDASDVEGYTPLHLAAYWGHLEIVEVLLKNGADVNAMDS
DGMTPLHLAAKWGYLEIVEVLLKHGADVNAQDKFGKTAFDISIDN
GNEDLAEILQKLN]

In Chain B (SEQ ID NO: 57) above, the double underlined indicates the region that corresponds to Subunit B of T33-51 (SEQ ID NO: 2, with a 5 amino acid truncation at the C-terminus, a deletion in the internal unstructured region); the passenger molecule (SEQ ID NO: 58) is italicized; the binder (MBP-specific DARPin, SEQ ID NO: 27, with a 4 amino acid N-terminal truncation) is bracketed; and the linker is in bold.

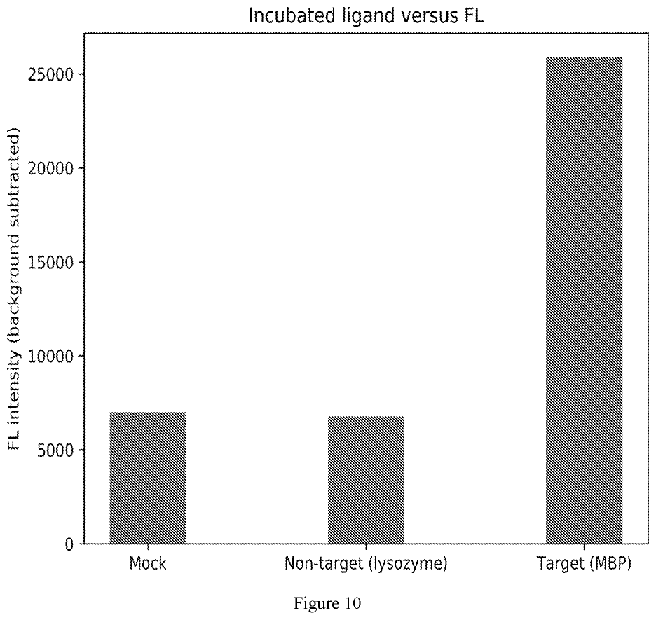

After expression of Chain B, a fluorophore, i.e., Oregon Green-maleimide (Invitrogen) bearing a thiol-reactive group, was covalently attached to the cysteine reside at amino acid position 2 using methods in the art. Briefly, Chain B was incubated at room temperature in pH 7.5 Tris-HCl buffer (comprising 150 mM NaCl, 2% glycerol) with a ten to twenty-fold molar excess of the fluorophore for at least two hours. All twelve copies of the B subunit of the assembled protein cages were found to contain the fluorophore attached thereto, which fluorophores are packaged within the interior cavities of the protein cages. Thus, the fluorophores are packaged together in close proximity (e.g., within about 50 Å) in the intact form of the LOC. Upon opening or disassembly of the protein cage, the fluorophores were free to separate away from each other, thereby resulting in an observable fluorescent signal as shown in FIG. 10 as their fluorescence was no longer self-quenched by the fluorophores being in close proximity to each other.

### REFERENCES

The following references are herein incorporated by reference in their entirety with the exception that, should the scope and meaning of a term conflict with a definition explicitly set forth herein, the definition explicitly set forth herein controls:
1. Bale, J. B. et al. Structure of a designed tetrahedral protein assembly variant engineered to have improved soluble expression: Designed Protein Tetrahedron. Protein Sci. 24, 1695-1701 (2015).
2. Cannon, K. A. et al. Design and structure of two new protein cages illustrate successes and ongoing challenges in protein engineering. Protein Sci. 29, 919-929 (2020).
3. Veith, A. et al. Substrate Pathways and Mechanisms of Inhibition in the Sulfur Oxygenase Reductase of Acidianus Ambivalens. Front. Microbiol. 2, (2011).
4. Arai, R., Ueda, H., Kitayama, A., Kamiya, N. & Nagamune, T. Design of the linkers which effectively separate domains of a bifunctional fusion protein. Protein Eng. Des. Sel. 14, 529-532 (2001).
5. Cole, C., Barber, J. D. & Barton, G. J. The Jpred 3 secondary structure prediction server. Nucleic Acids Res. 36, W197-W201 (2008).
6 Wetzel, S. K., Settanni, G., Kenig, M., Binz, H. K. & Plückthun, A. Folding and Unfolding Mechanism of Highly Stable Full-Consensus Ankyrin Repeat Proteins. J. Mol. Biol. 376, 241-257 (2008).
7 Zahnd, C. et al. A Designed Ankyrin Repeat Protein Evolved to Picomolar Affinity to Her2. J. Mol. Biol. 369, 1015-1028 (2007).
8 Monroe, N., Sennhauser, G., Seeger, M. A., Briand, C. & Grütter, M. G. Designed ankyrin repeat protein binders for the crystallization of AcrB: plasticity of the dominant interface. J. Struct. Biol. 174, 269-281 (2011).
9 Schroeder, T. et al. Specific Inhibition of Caspase-3 by a Competitive DARPin. Molecular Mimicry between Native and Designed Inhibitors. Structure 21, 277-289 (2013).
10. Teplyakov, A. et al. Conformational flexibility of an anti-IL-13 DARPin†. Protein Eng. Des. Sel. 30, 31-37 (2016).
11. Binz, H. K. et al. High-affinity binders selected from designed ankyrin repeat protein libraries. Nat. Biotechnol. 22, 575-582 (2004).
12. Hogbom, M., Eklund, M., Nygren, P.-A. & Nordlund, P. Structural basis for recognition by an in vitro evolved affibody. Proc. Natl. Acad. Sci. 100, 3191-3196 (2003).
13. Eigenbrot, C., Ultsch, M., Dubnovitsky, A., Abrahmsen, L. & Hard, T. Structural basis for high-affinity HER2 receptor binding by an engineered protein. Proc. Natl. Acad. Sci. 107, 15039-15044 (2010).
14. Grönwall, C. et al. Selection and characterization of Affibody ligands binding to Alzheimer amyloid beta peptides. J. Biotechnol. 128, 162-183 (2007).
15. Lindborg, M. et al. High-affinity binding to staphylococcal protein A by an engineered dimeric Affibody molecule. Protein Eng. Des. Sel. PEDS 26, 635-644 (2013).
16. Roy, R., Hohng, S. & Ha, T. A practical guide to single-molecule FRET. Nat. Methods 5, 507-516 (2008).
17. King, N. P. et al. Accurate design of co-assembling multi-component protein nanomaterials. Nature 510, 103-108 (2014).
18. Bale, J. B. et al. Accurate design of megadalton-scale two-component icosahedral protein complexes. Science 353, 389-394 (2016).
19. Wang, Z. et al. Structure of human ferritin L chain. Acta Crystallogr. D Biol. Crystallogr. 62, 800-806 (2006).
20. Ghosh, I., Hamilton, A. D. & Regan, L. Antiparallel Leucine Zipper-Directed Protein Reassembly: Application to the Green Fluorescent Protein. J. Am. Chem. Soc. 122, 5658-5659 (2000).
21. Cabantous, S., Terwilliger, T. C. & Waldo, G. S. Protein tagging and detection with engineered self-assembling fragments of green fluorescent protein. Nat. Biotechnol. 23, 102-107 (2005).
22. Hu, C.-D., Chinenov, Y. & Kerppola, T. K. Visualization of Interactions among bZIP and Rel Family Proteins in Living Cells Using Bimolecular Fluorescence Complementation. Mol. Cell 9, 789-798 (2002).

All scientific and technical terms used in this application have meanings commonly used in the art unless otherwise specified.

As used herein, a “ligand” refers to any naturally occurring or synthetic chemical or biomolecule (e.g., proteins, nucleic acids, carbohydrates, lipids, etc.) of interest. The ligands may be antigens, toxins, transmembrane proteins, receptors, cell markers, tumor markers, etc. The ligands may be anchored to a naturally occurring substrate, e.g., a cell surface, or a synthetic substrate, e.g., assay plate, well, or bead.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. In the context of a composition, “consists essentially of” a given ingredient means that the composition may comprise additional ingredients (which may exhibit biological or pharmaceutical activity) in addition the recited ingredient so long as the additional ingredients do not significantly affect the activity of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A ligand operable protein cage (LOC), which comprises a plurality of subunits bound together by non-covalent interactions, wherein at least one subunit comprises a sequence that has (a) at least 90% sequence identity over a comparison window of at least X-30 consecutive amino acid residues of a subunit protein having X number of amino acid residues, said subunit protein being of a known protein cage, and (b) a given binder covalently attached to the N-terminus or the C-terminus of the at least one subunit.

2. The LOC according to claim 1, wherein the known protein cage is selected from the group consisting of T33-51, T33-31, T33-21, T33-28, I53-40, I52-32, I32-28, ferritin, and sulfur oxygenase reductase.

3. The LOC according to claim 1, wherein the sequence of the subunit protein is selected from the group consisting of SEQ ID NOs: 1-20 and SEQ ID NOs: 38-56.

4. The LOC according to claim 1, wherein the sequence of the subunit protein is SEQ ID NO: 2 or SEQ ID NO: 34.

5. The LOC according to claim 1, wherein the given binder is an scFv, a Fab fragment, a Fab′ fragment, a F(ab′)2 fragment, an sdFv, an Fv, a protein comprising a set of CDRs of an antibody, a camelid nanobody, an affibody, an affilin, an affimer, an affitin, an alphabody, an anticalin, an avimer, a DARPin, an Fynomer, a Kunitz domain peptide, a monobody, or a nanoCLAMP known in the art.

6. The LOC according to claim 1, wherein the given binder is a DARPin or an affibody.

7. The LOC according to claim 6, wherein the DARPin comprises SEQ ID NO: 21.

8. The LOC according to claim 6, wherein the affibody comprises SEQ ID NO: 29.

9. The LOC according to claim 1, wherein the non-covalent interactions between the subunit and one of its adjacent subunits are disrupted when the given binder has its cognate ligand bound thereto.

10. The LOC according to claim 1, and further comprising a passenger molecule of interest covalently attached to the at least one subunit.

11. The LOC according to claim 1, wherein a passively packaged passenger molecule of interest is in the interior cavity of the LOC.

12. The LOC according to claim 10, wherein the passenger molecule and/or the passively packaged passenger molecule is a detectable label, a detection reagent, a theradiagnostic of interest, or a radiotheranostic of interest.

13. A method of delivering or releasing the passenger molecule claim 11, which comprises contacting the given binder with its cognate ligand.

14. An assay method which comprises using the LOC according to claim 1 to detect the presence or absence of the cognate ligand of the given binder in a sample by contacting the LOC with the sample and detecting the presence of absence of the cognate ligand bound to the given binder, wherein disassembly of the at least one subunit from the plurality of subunits indicates the presence of the cognate ligand.

15. A composition which comprises one or more LOCs according to claim 1.

16. A kit which comprises one or more LOCs according to claim 1 packaged together with the cognate ligand of the given binder and/or a reagent for detecting the disassembly of the at least one subunit from the plurality of subunits.
